SEQUENCE LISTING (58 sequences)

SEQ ID NO: 1 - protein, 12 aa, Artificial
  Fc effector peptide
CRWDGSWGEVRC

SEQ ID NO: 2 - protein, 12 aa, Artificial
  Fc effector peptide
CYWVGTWGEAVC

SEQ ID NO: 3 - protein, 7 aa, Artificial
  Fc effector peptide
XWXXXWG

SEQ ID NO: 4 - protein, 10 aa, Artificial
  Fc effector peptide
XXCPXCPXXP

SEQ ID NO: 5 - protein, 8 aa, Artificial
  Fc effector peptide
CLRSGXXC

SEQ ID NO: 6 - protein, 8 aa, Artificial
  Fc effector peptide
CLRSGRGC

SEQ ID NO: 7 - protein, 8 aa, Artificial
  Fc effector peptide
CLRSGLGC

SEQ ID NO: 8 - protein, 8 aa, Artificial
  Fc effector peptide
CLRSGAGC

SEQ ID NO: 9 - protein, 8 aa, Artificial
  Fc effector peptide
CLRSGSGC

SEQ ID NO: 10 - protein, 8 aa, Artificial
  Fc effector peptide
CLRSGRAC

SEQ ID NO: 11 - protein, 8 aa, Artificial
  Fc effector peptide
CLRSGANC

SEQ ID NO: 12 - protein, 8 aa, Artificial
  Fc effector peptide
CLRSGLHC

SEQ ID NO: 13 - protein, 8 aa, Artificial
  Fc effector peptide
CRRSGQGC

SEQ ID NO: 14 - protein, 8 aa, Artificial
  Fc effector peptide
CLYGDELC

SEQ ID NO: 15 - protein, 9 aa, Artificial
  Fc effector peptide
CFPVGRATC

SEQ ID NO: 16 - protein, 11 aa, Artificial
  Fc effector peptide
CSWIPGVGLVC

SEQ ID NO: 17 - protein, 11 aa, Artificial
  Fc effector peptide
CRRATAGCAGC

SEQ ID NO: 18 - protein, 11 aa, Artificial
  Fc effector peptide
CRSMVMLRVRC

SEQ ID NO: 19 - protein, 11 aa, Artificial
  Fc effector peptide
CGRVNTWLPQC

SEQ ID NO: 20 - protein, 11 aa, Artificial
  Fc effector peptide
CSAGRACCRYC

SEQ ID NO: 21 - protein, 21 aa, Artificial
  Fc effector peptide
CQDPICFCGADGACYCTSRNC

SEQ ID NO: 22 - protein, 25 aa, Artificial
  Fc effector peptide
CAWHYRFCGAAHSADGACREVFLVC

SEQ ID NO: 23 - protein, 11 aa, Artificial
  Fc effector peptide
CVVWMGFQQVC

SEQ ID NO: 24 - protein, 11 aa, Artificial
  Fc effector peptide
CWTSGARWRLC

SEQ ID NO: 25 - DNA, 26 bp, Artificial
  Primer
catccgccccaaagcttgcctccacc

SEQ ID NO: 26 - DNA, 61 bp, Artificial
  Primer
ggccccagcggccccggatccggccccgtcggccccgggcttgtgattcacgttgcagatg

SEQ ID NO: 27 - DNA, 61 bp, Artificial
  Primer
ggggccgacggggccggatccggggccgctggggccagcaacaccaaggtggacaagaaag

SEQ ID NO: 28 - DNA, 37 bp, Artificial
  Primer
tataataggatcccccacagtctcccctgttgaagct

SEQ ID NO: 29 - DNA, 22 bp, Artificial
  Primer
gtacaaaccacaacgcctgtag

SEQ ID NO: 30 - DNA, 21 bp, Artificial
  Primer
tcgaaagcaacgtgataaacc

SEQ ID NO: 31 - DNA, 20 bp, Artificial
  Primer
ccctcatagttagcgtaacg

SEQ ID NO: 32 - protein, 11 aa, Artificial
  Irrelevant insert
CGPGGTVGYTC

SEQ ID NO: 33 - DNA, 21 bp, Artificial
  Primer
atctctttgcagcctccatgg

SEQ ID NO: 34 - DNA, 21 bp, Artificial
  Primer
atgaaaccagacaggagttgg

SEQ ID NO: 35 - DNA, 30 bp, Artificial
  Primer
gagagagagaaagcttatctctttgcagcc

SEQ ID NO: 36 - DNA, 37 bp, Artificial
  Primer
gagagagagaggatccgggcccatgaaaccagacagg

SEQ ID NO: 37 - DNA, 30 bp, Artificial; identical to SEQ ID NO: 35
  Primer

SEQ ID NO: 38 - DNA, 38 bp, Artificial
  Primer
gagagagagaggatccactcaccatgaaaccagacagg

SEQ ID NO: 39 - protein, 21 aa, Artificial
  Fc effector peptide
CQDPICFCGADGACYCTSRNC

SEQ ID NO: 40 - protein, 25 aa, Artificial
  Fc effector peptide
CAWHYRFCGAAHSADGACREVFLVC

SEQ ID NO: 41 - DNA, 50 bp, Artificial
  Primer
ttactcgcggcccagccggccatggcccaggtccaactgcagcagcctgg

SEQ ID NO: 42 - DNA, 38 bp, Artificial
  Primer
tagcgtacctcgagtgaggagactgtgagagtggtgcc

SEQ ID NO: 43 - DNA, 86 bp, Artificial
  Primer
atagtcaactcgagggtggtggtggttctgggggcggaggatccggcgggggagggtcagagctccaggctgttgtgactcaggaa

SEQ ID NO: 44 - DNA, 37 bp, Artificial
  Primer
tttgttctgcggccgcacctaggacagtcagtttggt

SEQ ID NO: 45 - DNA, 22 bp, Artificial
  Primer
tcgaaagcaagctgataaaccg

SEQ ID NO: 46 - protein, 26 aa, Artificial
  C-terminal part of ani-NIP SCFV fragments, c-my/his tag
VLGAAAGSEQKLISEEDLNSHHHHHH

SEQ ID NO: 47 - protein, 17 aa, Artificial
  C-terminal part of anti-NIP SCFV fragments, vector with SFI1 site
VLGAAAADGAGSGAAGA

SEQ ID NO: 48 - protein, 27 aa, Artificial
  C-terminal part of anti-NIP SCFV fragments, PSG1 with insert DER1
VLGAAAADGACRWDGSWGEVRCGAAGA

SEQ ID NO: 49 - protein, 27 aa, Artificial
  C-terminal part of anti-NIP SCFV fragments PSG1 with insert DERIV
VLGAAAADGACYWVGTWGEAVCGAAGA

SEQ ID NO: 50 - DNA, 19 bp, Artificial
  Primer
caatatttgcggccgcggc

SEQ ID NO: 51 - DNA, 18 bp, Artificial
  Primer
gacccatctagataggcc

SEQ ID NO: 52 - DNA, 64 bp, Artificial
  DNA insert containing fuse5 compatible sfi1 sites
caatatttgcggccgcggccgacggggccggatccggggccgctggggcctatctagatgggtc

SEQ ID NO: 53 - DNA, 299 bp, Artificial
  PCR template
attcacctcgaaagcaagctgataaaccgatacaattaaaggctccttttggagcctttttttttggagattttcaacgtgaaaaaattattattcgcaattcctttagttgttcctttctattctcactcggccgacggggccggccgctggggccgaaactgttgaaagttgtttagcaaaacctcatacagaaaattcatttactaacgtctggaaagacgacaaaactttagatcgttacgctaactatgagggctgtctgtggaatgctacaggcgttgtggtttgtactggtg

SEQ ID NO: 54 - protein, 16 aa, Artificial
  sequence encoded by plasmid pFab SfiIL6
KPGADGAGSGAAGASN

SEQ ID NO: 55 - DNA, 48 bp, Artificial
  Sequence of plasmid pFab SfiIl6
aagcccggggccgacggggccggatccggggccgctggggccagcaac

SEQ ID NO: 56 - protein, 8 aa, Artificial
  Fc effector peptide
CLRSGXXC

SEQ ID NO: 57 - protein, 12 aa, Artificial
  Fc effector peptide
CYWVGTWGEAVC

SEQ ID NO: 58 - protein, 5 aa, Artificial
  Control peptide
PFABK

(19) United States
(12) Patent Application Publication
(10) Pub. No.: US 2004/0101905 A1
(43) Pub. Date: May 27, 2004

(54) Product

(76) Inventors: Ole Henrik Andre Brekke, Oslo (NO); Vigdis Lauvrak, Oslo (NO); Inger Sandie, Oslo (NO)

Correspondence Address: CANTOR COLBURN, LLP, 55 GRIFFIN ROAD SOUTH, BLOOMFIELD, CT, 06002

(21) Appl. No.: 10/433,452

(22) PCT Filed: Nov. 30, 2001
(86) PCT No.: PCT/GB01/05301

(30) Foreign Application Priority Data
Dec. 1, 2000 (GB) 0029407.4

(51) Int. Cl. G01N 33/53; C07K 16/28

(52) U.S. Cl. 435/7.1; 530/388.22

(57) ABSTRACT

The present invention relates to binding molecules comprising (i) one or more polypeptides which form a binding site capable of binding a target molecule and (ii) an Fc effector peptide displaying one or more effector functions associated with the constant region (Fc) of an immunoglobulin heavy chain. The invention further relates to novel Fc effector peptides and nucleic acid molecules encoding said binding molecules and Fc effector peptides. The invention further relates to therapeutic uses of said binding molecules and pharmaceutical compositions containing said binding molecules.

The present invention relates to binding molecules comprising one or more binding sites, preferably antigen binding sites, capable of binding target molecules, and an Fc effector peptide displaying one or more of the effector functions associated with the constant region (Fc) of an immunoglobulin heavy chain. The invention further relates to nucleic acids encoding said binding molecules and effector peptides, host cells expressing said binding molecules and effector peptides and methods of producing and uses of said binding molecules and effector peptides.

Recombinant antibodies and their fragments represent over 30% of all biological proteins undergoing clinical trials for diagnosis and therapy (Hudson P J. Curr Opin Immunol. 1999 October; 11(5):548-57. Review). Since the development of mouse monoclonal antibodies (mAbs) from hybridomas (Kohler and Milstein, 1975 Nature 256, 495-7) mAbs have been constructed for use in therapy. However, the clinical potential has been hampered by their immunogenicity. Evidently, clinical mAbs should be as “human” as possible, and genetic engineering has allowed development of chimaeric Abs (Morrison et al., Proc Natl Acad Sci USA 1984, 81:68851-5) and CDR-grafted Abs (Jones et al. Nature 1986, 321: 522-5; Riechmann et al. Nature 1988, 332: 323-7;).

Several advances made during the past years will probably further facilitate the development of therapeutic antibodies. Most notably, significant progress has been made in the rapid isolation of high affinity human antibodies from phage display libraries (Griffiths and Duncan, Curr Opin Biotechnology 1998, 9: 102-8. Review) and by immunization of transgenic mice (Lonberg N. et al. Nature 1994, 368: 856-9).

Antibodies are particularly attractive tools for use in diagnosis and therapy due to the fact that they show specific targeting by virtue of their specific interaction with a particular antigen. Thus, antibodies can be targeted to particular target cells, organs, tissues, foreign organisms, or other body sites etc. by selecting an antibody specific for a particular antigen found on the target cells or organisms or in the body sites in question. Other types of molecule (i.e. non-antibody molecules) also show specific binding to binding partners, for example receptors, enzymes, hormones, ligands, antigens, cytokines and enzyme substrates. Thus, any of these specific binding partners may also be used to target entities to specific cells, tissues, foreign organisms, body sites etc., providing the cognate member of the particular specific binding partner chosen is expressed on the cells, organisms, or in the body site in question.

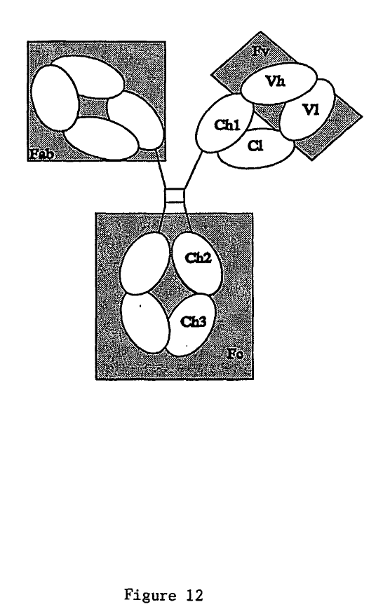

Antibodies (Immunoglobulins, (FIG. 12)) exhibit at least two functions in the immune system. They bind antigens and eliminate these via the immunoglobulin effector functions such as activation of the complement system or interaction with cellular receptors (Fc receptors) on phagocytic cells such as macrophages, and other cells such as leukocytes, platelets and placental trophoblasts. Immunoglobulins consist of heavy and light chains, the N-terminal domains of which form a variable domain responsible for the binding of antigen. The variable domain is associated with a constant or C-terminal domain which defines the class of immunoglobulin. Thus, as can be seen from FIG. 12, a typical immunoglobulin light chain comprises one variable domain (V_{L}) and one constant domain (C_{L}) and a typical heavy chain comprises one variable domain (V_{H}) and three constant domains (C_{H}1, C_{H}2 and C_{H}3). It is the so called Fc region of the heavy chain which is responsible for the immunoglobulin effector functions. This region is made up of the C_{H}2 and C_{H}3 domains of the heavy chain.

The minimal antibody fragments, responsible for antigen binding may be composed of the variable domains of the light and heavy chains, e.g. Fv fragments which comprise the V_{H} and V_{L} domains, or two variable and two constant domains of the heavy and light chains, e.g. Fab fragments, which comprise the V_{H}, V_{L}, C_{H}1 and C_{L} domains (see FIG. 12). Such antibody fragments can be successfully expressed in E. Coli as well as eukaryotic cells (Kipriyanov S M and Little M. Mol. Biotechnology 1999, September; 12(2):173-201. Hudson P J. Curr Opin Biotechnol. 1998 August; 9(4):395-402). In addition, Fv fragments can be produced as so called “single chain” antibody fragments by arranging the V_{H} and V_{L} domains as a single polypeptide joined by a peptide linker.

The common factor with regard to all these minimal antibody fragments is that the constant Fc region of the native immunoglobulin heavy chain is absent. Thus, it can be seen that only intact immunoglobulins (and not minimal antibody fragments) exhibit immunoglobulin effector functions.

It can be seen that the production of intact antibodies or antibody fragments which display immunoglobulin effector functions, or indeed the production of non-antibody based targeting moieties displaying immunoglobulin effector functions, would be advantageous in that they would more closely mimic native antibodies. However, in the production of intact and active immunoglobulins it is known that glycosylation of the Fc part of the heavy chain is a crucial event (Tao and Morrison, J. Immunol 1989, 143:2595-601, Jefferis et al., Immunol Rev, 1998 163, 59-76. Review). In prokaryotes, such as E. Coli, glycosylation does not occur. Thus, intact and active immunoglobulins with respect to natural effector functions can not be expressed in E. Coli, which is disadvantageous. Moreover, the size of intact antibodies makes them difficult to produce using conventional expression methods in eukaryotic hosts where glycosylation might occur. In addition, glycosylation is species specific, meaning that if for example a human intact antibody is produced in a cell type of a different species, although the antibodies might be active in terms of effector functions, they are likely to elicit an immune response due to the “foreign” glycosylation pattern. This may in turn result in the unwanted elimination of the administered antibody. Finally, the size of intact antibodies means that even if the antibodies are produced displaying immunoglobulin effector function and are targeted to the correct location in a subject, tissue penetration is unlikely to is occur. This is where the antibody fragments with their smaller sizes can be advantageous.

The sites on Fc regions which are involved in the binding of effector molecules are in many cases not completely characterized, and where information is available, they consist of patches of amino acids that are located far apart in the linear polypeptide chain. Consequently, it was believed that only complete non linear heavy chain Fc regions, consisting of both constant domains (i.e. the C_{H}2 and C_{H}3 domains), glycosylated and preferably paired such that two identical halves are connected by a hinge region amino terminally, could have natural effector functions.

Surprisingly however and contrary to expectation it has been found that relatively short linear or cyclic peptides can give rise to immunoglobulin effector functions such as complement activation and/or Fc receptor binding. These Fc effector peptides can be conjugated to, fused to or associated with minimal antibody fragments to result in a recombinant antibody molecule which displays both specific antigen binding and effector function. These Fc effector peptides can also be conjugated to, fused to or associated with any member of a specific binding pair, thereby allowing the Fc effector peptide to be targeted to locations where, the other member of the specific binding pair is found.

The relatively small size of the Fc effector peptide-specific binding pair member conjugate (referred to herein as a “binding molecule”) will facilitate tissue penetration and these entities can be used as therapeutics in diseases where the stimulation of immunoglobulin effector function is useful. Moreover, the Fc effector peptides of the present invention do not require glycosylation to exhibit immunoglobulin effector activities and thus can be produced on a large scale in prokaryotic hosts such as E. coli. The lack of glycosylation and the fact that the Fc effector peptides mimic the activity of naturally occuring immunoglobulins and utilise the body's own elimination system for target destruction means that they are likely to be less immunogenic than other antibody or non-antibody based therapeutic molecules.

Thus, viewed from one aspect the present invention provides a binding molecule comprising (i) one or more polypeptides which form a binding site capable of binding a target molecule and (ii) an Fc effector peptide displaying one or more effector functions associated with the constant region (Fc) of an immunoglobulin heavy chain.

The binding molecules of the invention, although they may mimic naturally occurring or native binding molecules, e.g. native antibodies, do not correspond to naturally occurring or native binding molecules.

The polypeptides which form the binding site can be derived from any source appropriate to the application to which the binding molecule is designed to be put and may be derived from for example an antibody, receptor, hormone, enzyme, antigen, cytokine or other ligand. Preferably all or part of the polypeptides are derived from an antibody molecule (an immunoglobulin molecule) or a derivative thereof, particularly natural or modified variable and or constant domains of an antibody molecule, and the binding site is an antigen binding site. Thus, the polypeptides making up the binding site may be derived from one or more native antibody domains, or may be polypeptide sequences which are substantially homologous to such domains or functional derivatives or variants thereof (e.g. as defined herein) which may be produced for example by one or more of single or multiple amino acid addition, deletion or substitution. Said “derivatives” of the polypeptides include polypeptides which have been modified in any appropriate way but still retain the appropriate binding function. Indeed, the binding function may well be improved by such derivatization. Said “derivatives” thus include peptides which are “substantially homologous” to the native polypeptide domains according to the definition provided herein, i.e. also include functionally equivalent variants and related sequences as defined herein. In addition, said antibody domains may be wholly or partially synthetic, e.g. may not correspond to or derive from naturally occurring antibody immunoglobulin polypeptide domains but comprise one or more random or semi-random peptide/amino acid sequences.

Where the polypeptides form an antigen binding site, the polypeptides making up said site generally comprise variable and/or constant domains from heavy and light chains of antibodies which may be derived from the same or different native antibody molecule, or may be substantially homologous to such native domains or may be variants, derivatives or wholly or partially synthetic versions thereof (as outlined above). More preferably the antibody derived polypeptides, derivatives, synthetic molecules, etc., are antibody fragments such as single chain Fv fragments (scFv), Fv or Fab fragments. The type of variable and constant domains of antibody molecules making up such antibody fragments are well known in the art and are described above and also in FIG. 12. Such preferred binding molecules which comprise a binding site composed of an antibody fragment associated with an Fc effector peptide are sometimes referred to herein as “pepbodies”. Especially preferred pepbodies contain a scFv fragment or a Fab fragment as a binding site.

It will be appreciated that, depending on the type of antibody fragment chosen, the binding site will be made up of one or several polypeptide chains which associate with each other to form the antigen binding site e.g. by covalent or any other type of interaction such as hydrophobic or ionic interactions or sulphide bridge linkages. For example, in the case of scFv fragments, the variable domains from the heavy (V_{H}) and light (V_{L}) chains of one or more immunoglobulins which make up the antigen binding site are connected by a peptide linker and form part of the same single polypeptide chain. In the case of Fv fragments, the V_{H} and V_{L} domains are generally provided on separate polypeptide chains and the domains associate together via non di-sulphide bonding to form the antigen binding site. Similarly for Fab fragments, the V_{H} and C_{H}1 domains and the V_{L} and C_{L} domains are generally provided on two separate polypeptide chains which associate to form the antigen binding site.

Similarly where the binding site is not an antigen binding site derived from an antibody but a binding site derived from a non-antibody based source e.g. from a receptor, hormone, enzyme, antigen or other ligand there may, if appropriate, be more than one polypeptide chain making up the binding site, depending on the structure of the particular binding site chosen. For example, if in the native state, the binding site is made up of more than one polypeptide chain, then these chains can either be linked on a single polypeptide chain (as for scFv above) or provided by separate chains which can associate together to form the binding site.

A binding molecule of the invention may, if desired, comprise than one binding site, which can be for the same or different target molecules. Thus, the binding molecules may be multimeric (e.g. dimeric or trimeric etc.) in terms of the binding sites available to bind target molecules. So called “bispecific” antibodies or antibody fragments which have antigen binding sites specific for different targets are known in the art and are sometimes referred to as bispecific diabodies (Kontermann et al., Nat Biotechnol, 1997, 15(7): 629-31; Holliger et al. Nat Biotechnol, 1997 15(7):632-6) or triabodies (Kipriyanov S M. et al., J Mol Biol 1999 Oct. 15; 293(1):41-56). These bispecific antibodies can be constructed in bacteria by joining the variable domains of two antibodies through short polypeptide linkers. These chains are co-expressed in the same cell and associate to form heterodimers with two antigen-binding sites on the same molecule. Such bispecific diabodies or triabodies, and indeed any other antibody derived molecule with more than one antigen binding site (for the same or different targets) can be used as binding sites in the binding molecules of the present invention.

The binding site will have an ability to interact with a target molecule which will preferably be another polypeptide, but may be any target, e.g. a carbohydrate, lipid or nucleic acid containing molecule. Preferably the interaction will be specific. The binding site may derive from the same source or different source to the Fc effector peptide. In preferred embodiments where the binding site is an antibody derived antigen binding site the target will be the antigen recognised by the binding site or a receptor with a soluble ligand for which the antibody competes.

“polypeptide” as used herein refers to oligo and polypeptides including proteins, protein fragments, etc. The polypeptides making up the binding site for the target molecule can be of any appropriate length and composition providing that a functional binding site can be formed.

“peptide” as used herein refers to a relatively short amino acid sequence, e.g. up to 100 residues, preferably 5 to 70 residues, more preferably 5 to 50 residues, more preferably 6 to 30 residues, most preferably 6 to 20 or 6 to 25 residues and especially preferably 6 to 15 residues. The term “peptide” is used herein in connection with the term “Fc effector peptide”. Such peptides are of the lengths as defined above and are required to display one or more of the natural effector functions associated with the constant region (Fc) of an intact whole immunoglobulin molecule. Such Fc effector peptides can therefore be regarded as Fc region mimics.

The Fc effector peptides of the present invention can correspond to or comprise short active fragments of the Fc region as found in intact naturally occurring immunoglobulins, e.g. active fragments of the C_{H}2 and C_{H}3 domains of a particular class of immunoglobulin molecule. The Fc effector peptides of the present invention do not however correspond to the complete Fc region, i.e. do not contain both the C_{H}2 and C_{H}3 domains of an intact immunoglobulin. Indeed, Fc effector peptides of the present invention do not contain complete C_{H}2 and/or C_{H}3 domains of an intact immunoglobulin, only active fragments thereof. The Fc effector peptides can be derived from the same or different source as the polypeptides making up the binding site. However, in a preferred embodiment of the invention the Fc effector peptides do not correspond to amino acid sequences as found in naturally occurring Fc regions (e.g. are synthetic peptides, for example peptides comprising random or semi-random peptide sequences), although in this embodiment some amino acids making up the Fc effector peptides may correspond to amino acids which are found in native immunoglobulin molecules, i.e. parts of the Fc effector peptides may correspond to amino acids which are found in native immunoglobulin molecules. Preferably the Fc effector peptides are linear and do not require glycosylation to exhibit immunoglobulin effector function. Alternatively, preferred Fc effector peptides may be cyclic, e.g. by virtue of containing covalent bonds between one or more pairs of cysteine residues.

As mentioned above the Fc effector peptides display one or more of the natural effector functions associated with the Fc region of an intact immunoglobulin. The Fc regions are constant within classes of immunoglobulin but vary from class to class. Indeed, it is the nature of the Fc region which forms the basis of immunoglobulin classification. Thus, there are different Fc regions associated with IgG1, IgG2, IgG3, IgG4, IgA, IgE, IgM and IgD immunoglobulins, although the Fc regions associated with the four sub-classes of IgG in humans (i.e. IgG1, IgG2, IgG3 and IgG4) are very similar (over 90% homology). Depending on the type of Fc region present in a molecule different biological effector functions may be present. However, the most common types of effector activity are the ability to bind Fc receptors and the ability to activate complement by binding to proteins in the complement pathway. Thus, Fc effector peptides which display either or both of these activities are preferred.

Where the Fc effector peptides have the ability to activate complement, this is generally manifested in an ability to bind proteins which are part of the C1 protein complex. The C1 protein complex is made up of the proteins C1q, C1r and C1s and is the first component of the classical pathway of complement activation. C1q binding to aggregated IgG molecules via the Fc effector regions of the IgG results in activation of the classical pathway of complement leading to one or more of the effects of target cell lysis, opsonisation (i.e. uptake) of the immune complex, cytokine release, increased inflammation and eventually clearance of the immune complex. The induction of such activities by Fc effector peptides of the invention in combination with the ability to target such activities to specific cells, foreign organisms or body sites (via for example the binding site component of the binding molecule) can be harnessed for use in therapy (as discussed in more detail below). Preferably the complement activating Fc effector peptides will bind the C1q protein. Some examples of effector peptides which can bind C1q are disclosed in Lauvrak et al (Biol. Chem. 1997, 378(12): 1509-19) and any of these can be used as components of binding molecules in accordance with the present invention.

Preferred Fc effector peptides which have the ability to bind the C1q protein and activate complement consist of or comprise the amino acid sequences CRWDGSWGEVRC or CYWVGTWGEAVC, or functional fragments thereof, or a sequence which is substantially homologous to these sequences or fragments. Further especially preferred Fc effector peptides which can activate complement consist of or comprise the amino acid sequences h/RWXXXWG or R/KP/DCPS/TCPXXP (h is a large hydrophobic amino acid, e.g. phenylalanine: F, tyrosine: Y, or tryptophane:W, X is a less conserved or variable amino acid and underlined residues are invariant amino acids), or functional fragments thereof, or a sequence which is substantially homologous to these sequences or fragments.

Fc effector peptides may also activate complement by binding to protein components in the complement cascade other than members of the C1 complex. For example the Fc effector peptides for use in the present invention may activate complement by interacting with the C3 complement protein.

Where the Fc effector peptides have the ability to bind Fc-receptors these receptors include Fc-gamma receptors such as FcγRI (CD64), FcγRII (CD32, which exists as two sub-types—FcγRIIa and FcγRIIb) and FcγRIII (CD16, which also exists in two sub-types—FcγRIIIa and FcγRIIIb), Fc-epsilon receptors such as FcεRI, the poly Ig receptor (pIgR) which can bind the Fc regions of polymeric forms of IgA and IgM and result in their transcytosis through epithelia to the apical surface and the neonatal Fc-receptor (FcRn) which can bind the Fc region of IgG immunoglobulins and result in their transportation to the neonate and increased serum half-life. Fc effector peptides with the ability to bind one or more of these Fc-receptors are preferred.

In general, the induction of an immune response depends on the antibody mediated binding of antigens to cellular Fc receptors and the subsequent initiation of cellular effector functions of the immune system. When the Fc receptor is on an effector cell the binding can trigger the effector cells to kill target cells to which the antibodies are bound via the variable (v) regions (i.e. the antigen binding site region). Also opsonisation (uptake) of the immune complexes and the release of cytokines can be stimulated by binding the Fc receptors. In the case of IgG antibodies once an immunogenic particle is crosslinked by IgG, the Fc part of IgG crosslinks FcγRs on the cell surface of immunocompetent cells and triggers the immune response. As outlined above, there are three classes of Fc gamma receptors, FcγRI (CD 64, K_{A}≈10⁸−10⁹ M⁻¹), FcγRII (CD 32, K_{A}<10⁷ M⁻¹) and FcγRIII (CD 16, K_{A}≈10⁷ M⁻¹). Only FcγR1 is able to bind IgG in a monomeric form and the affinity of FcγRI receptors compared to the immunoglobulin receptors FcγR11 and FcγRIII is high. The high affinity receptor FcγRI is constitutively expressed on monocytes, macrophages and dendritic cells and expression can be induced on neutrophils and eosinophils, and thus these cells can be recruited to a target site by use of an Fc effector peptide which binds FcγRI.

The FcγRIIa receptor is found on macrophages, monocytes and neutrophils and the FcγRIIb receptor is found on B-cells, macrophages, mast cells and eosinophils. The FcγRIIIa receptor is found on NK cells, macrophages, eosinophils, monocytes and T cells and the FcγRIIIb receptor is highly expressed on neutrophils. Again, these various cell types can be recruited to a target site depending on the ability of the Fc effector peptide to bind the various types of Fcγ receptors.

Thus, the Fc effector peptides of the present invention may display one or more of the effector functions such as binding to Fc receptors and activating complement and may bind one or more class or sub-class of Fc receptors. More than one Fc effector peptide displaying the same type of effector function may be used in the binding molecules of the invention. Alternatively, the binding molecules of the invention can be constructed to combine more than one effector function by including in said binding molecules more than one (i.e. two or more) Fc effector peptides which individually display the distinct and required effector functions. Such binding molecules with multiple Fc effector peptides can for example be obtained by conjugating, fusing or associating two or more different Fc effector peptides which exhibit differing effector functions to the polypeptides which form the binding site of the binding molecules.

Due to the fact that the Fc effector peptides of the present invention display one or more defined effector functions, the types of immunoglobulin effector functions stimulated by the binding molecules of the present invention can be selected depending on the final use to which the molecules are to be put. For example in some situations, e.g. for some therapeutic uses, it may be desirable to have only Fc receptor mediated effector functions and no complement activation, or vice versa, or Fc effector peptides which only bind and activate effector functions associated with a certain class or sub-class of Fc receptors. Fc effector peptides with these discriminatory activities can be selected and produced, for example by methods as described herein. Thus, the effector functions of the Fc effector peptides of the present invention can differ from what is found for natural antibodies, in that the effector peptides can display discriminatory binding to Fc receptors and complement proteins. For example, if desired it is possible to select an effector peptide which can bind the FcγRI receptor and thereby mediate FcγRI mediated effector functions and which cannot bind the FcγRII or FcγRIII receptors or activate complement. Natural IgG antibodies would not generally discriminate between Fcγ receptor subclasses and complement protein binding in this way. Another example is effector peptides which can bind both FcαR and FcγR.

The Fc effector peptide can be located at any position in the binding molecule provided that the location does not result in the loss of the particular effector function associated with the Fc effector peptide or the interference with the ability of the binding site formed by the polypeptides (e.g. the antigen binding site) to bind the target molecule (e.g. the target antigen). For example the Fc effector peptide can be located at or near the N-terminus or C-terminus of one of the polypeptides which form the binding site for a target molecule or can be located anywhere between said N-terminus and C-terminus (for example can be inserted within one of the polypeptides forming the binding site). In the cases where the binding sites are made up of light and heavy chains of antibody molecules, the Fc effector peptides may be inserted within the antibody polypeptides, e.g. can be inserted in the loop regions of the antibody fragments, providing the ability to bind antigen is not adversely effected. In particular, it is shown in the attached Examples that an Fc effector peptide can be inserted into the loop between the beta strands f and g of a CH 1 domain of a Fab fragment without interfering with the ability of the Fab fragment to bind antigen. Such locations of Fc effector peptide can be regarded as preferred.

In addition, as mentioned above, more than one Fc effector peptide can be included in the binding molecule, e.g. more than one Fc effector peptide can be associated with or fused to the polypeptides which form a particular binding site. Such multiple Fc effector peptides can be located in the same region of the binding molecule by, for example, joining the effector peptides together with one or more linker peptides. Alternatively, they can be associated with different parts of the binding molecule.

If the binding molecule is multimeric in terms of binding sites, i.e. contains more than one binding site for one or more distinct target molecules then one or more Fc effector peptides may be associated with each polypeptide or “set” of polypeptides making up the binding site. Alternatively, the Fc effector peptides may be associated with only one of the binding sites (if the binding molecule is dimeric) or one or two of the binding sites (if the binding molecule is trimeric), etc.

In all the above described embodiments, preferably the Fc effector peptides are located at or near the C-terminus of one or more of the polypeptides which form the binding site. In alternative preferred embodiments the Fc effector peptides are inserted into the loop regions of one or more of the polypeptides which form the binding site, for example into the loop regions of domains of antibody fragments. In particular, where the binding site is a Fab fragment, the Fc effector peptides are preferably inserted into the loop regions of the CH1 domain, more preferably into the loop between the beta strands f and g of the CH 1 domain.

Fc effector peptides as described herein which have the ability to bind one or more Fc-receptors (i.e. Fc receptor binding effector peptides) provide a further aspect of the present invention.

Preferred Fc effector peptides of the present invention which have the ability to bind Fc receptors comprise or consist of the amino acid sequences CLRSGXXC (where X is a variable amino acid), for example comprise or consist of the sequences CLRSGRGC, CLRSGLGC, CLRSGAGC, CLRSGSGC, CLRSGRAC, CLRSGANC, or CLRSGLHC (see Table 1), or functional fragments thereof, or a sequence which is substantially homologous to these sequences or fragments. Further especially preferred sequences of Fc effector peptides which have the ability to bind Fc receptors comprise or consist of the amino acids CRRSGQGC, CLYGDELC, CFPVGRATC (see Table 1), or functional fragments thereof, or a sequence which is substantially homologous to these sequences or fragments. Further especially preferred sequences of Fc effector peptides which have the ability to bind Fc receptors comprise or consist of the amino acid sequences CSWIPGVGLVC, CRRATAGCAGC, CRSMVMLRVRC, CGRVNTWLPQC or CSAGRACCRYC (see Table 2), or functional fragments thereof, or a sequence which is substantially homologous to these sequences or fragments. These Fc effector peptides have been shown to bind the FcγRI receptor which is a high affinity IgG receptor found on a number of cell types involved in the immune response, as discussed above.

Other preferred Fc receptor binding effector peptides comprise or consist of the amino acid sequences CQDPICFCGADGACYCTSRNC, CAWHYRFCGAAHSADGACREVFLVC, CVVWMGFQQVC or CWTSGARWRLC, or functional fragments thereof, or a sequence which is substantially homologous to these sequences or fragments. These Fc effector peptides have been shown to bind the poly Ig receptor.

Native IgA and IgM immunoglobulins are transcytosed through epithelia by the poly Ig receptor. Poly Ig receptor binds to the Fc region of both these antibody classes, provided they are polymers of two (IgA) or five (IgM) monomers and have bound J-chain. Poly Ig receptor-antibody complexes are transcytosed from the basolateral to the apical side of epithelium mucosal surfaces after which the extracellular portion of the poly Ig receptor (the secretory component, SC) stays bound to the antibody and stabilizes the antibody molecule against proteolytic degradation. The exact nature of the binding sites on polymeric IgA and IgM for the poly Ig receptor is still a matter of dispute, but it is believed to involve both a portion of the antibody Fc region as well as a portion of the J chain. For this reason it has been considered extremely difficult to produce antibody derived molecules with the ability to be transcytosed via the poly Ig receptor unless such molecules contain intact Fc regions of polymeric antibodies correctly bound to J-chain. Thus, the identification of short linear or cyclic peptides which have this ability, such as those described above, is very surprising. As will be described in more detail in the Examples, such short linear or cyclic peptides displayed as fusion proteins with protein III on the surface of phage give phage particles the ability to be transcytosed. Thus, the binding molecules of the invention fused to such peptides will be transported/transcytosed to mucous membranes. In particular, the pepbodies of the invention that contain a binding site fused to such peptides, will be transported/transcytosed to mucous membranes where they will mimic the action of normal antibodies.

Nucleic acid molecules comprising or consisting of nucleic acid sequences encoding one or more Fc effector peptides which display one or more effector functions associated with the constant region (Fc) of an immunoglobulin heavy chain and which preferably have the ability to bind Fc receptors and/or the ability to activate complement, and especially nucleic acid molecules comprising sequences encoding the preferred amino acid sequences as defined above also form part of the present invention, as do nucleic acid molecules comprising or consisting of nucleic acid sequences which are degenerate to, substantially homologous with, or which hybridise with nucleic acid sequences which encode is Fc effector peptides which have the ability to bind Fc receptors and/or the ability to activate complement (and especially the preferred sequences as defined above), or which hybridise with the sequence complementary to such an encoding sequence. Fragments of such nucleic acid molecules encoding a functionally active product are also included.

Nucleic acid molecules comprising nucleic acid sequences which encode one or more polypeptides which form all or part of a binding site capable of binding a target molecule, together with nucleic acid sequences which encode one or more Fc effector peptides displaying one or more effector functions associated with the constant region (Fc) of an immunoglobulin heavy chain, and which preferably have the ability to bind Fc receptors and/or the ability to activate complement, form yet further aspects of the invention. Nucleic acid molecules comprising or consisting of nucleic acid sequences which are degenerate to, substantially homologous with, or which hybridise with nucleic acid sequences which encode said sequences or which hybridise with the sequence complementary to such an encoding sequence are also included within the scope. Fragments of such nucleic acid molecules encoding a functionally active product are also included. Preferred Fc effector peptides which are encoded by said nucleic acid molecules are as described herein.

“Functionally active-product” as used herein refers to a product encoded by said nucleic acid sequence which exhibits Fc receptor binding activity and/or the ability to activate complement.

“Degenerate” as used herein in connection with a nucleic acid sequence refers to nucleic acid sequences which contain base changes (i.e. nucleotide changes) that do not cause a change in the encoded amino acid sequence.

“Substantially homologous” as used herein in connection with an amino acid or a nucleic acid sequence includes those sequences having a sequence homology or identity of approximately 60% or more, e.g. 70%, 80%, 90%, 95%, 98% or more with a particular sequence and also functionally equivalent variants and related sequences modified by single or multiple base or amino acid substitution, addition and/or deletion. By “functionally equivalent” in this sense is meant nucleotide sequences which encode functionally active Fc effector peptides which have the ability to bind Fc receptors and/or the ability to activate complement, as appropriate, or amino acid sequences comprising such functionally active peptides. Such functionally equivalent variants may include synthetic or modified amino acid or nucleotide residues providing the function of the molecule as a whole is retained.

Homology may be assessed by any convenient method. However, for determining the degree of homology between sequences, computer programs that make multiple alignments of sequences are useful, for instance Clustal W (Thompson, J. D., D. G. Higgins, et al. (1994). “CLUSTAL W: Improving the sensitivity of progressive multiple sequence alignment through sequence weighting, position-specific gap penalties and weight matrix choice”. Nucleic Acids Res 22: 4673-4680). Programs that compare and align pairs of sequences, like ALIGN (E. Myers and W. Miller, “Optical Alignments in Linear Space”, CABIOS (1988) 4: 11-17), FASTA (W. R. Pearson and D. J. Lipman (1988), “Improved tools for biological sequence analysis”, PNAS 85:2444-2448, and W. R. Pearson (1990) “Rapid and sensitive sequence comparison with FASTP and FASTA” Methods in Enzymology 183:63-98) and gapped BLAST (Altschul, S. F., T. L. Madden, et al. (1997). “Gapped BLAST and PSI-BLAST: a new generation of protein database search programs”. Nucleic Acids Res. 25: 3389-3402) are also useful for this purpose. Furthermore, the Dali server at the European Bioinformatics institute offers structure-based alignments of protein sequences (Holm, J. of Mol. Biology, 1993, Vol. 233: 123-38; Holm, Trends in Biochemical Sciences, 1995, Vol 20: 478-480; Holm, Nucleic Acid Research, 1998, Vol. 26: 316-9).

By way of providing a reference point, sequences according to the present invention having 60%, 70%, 80%, 90%, 95% homology etc. may be determined using the ALIGN program with default parameters (for instance available on Internet at the GENESTREAM network server, IGH, Montpellier, France).

Sequences which “hybridise” are those sequences binding (hybridising) under non-stringent conditions (e.g. 6×SSC, 50% formamide at room temperature) and washed under conditions of low stringency (e.g. 2×SSC, room temperature, more preferably 2×SSC, 42° C.) or conditions of higher stringency (e.g. 2×SSC, 65° C.) (where SSC=0.15M NaCl, 0.015M sodium citrate, pH 7.2).

Generally speaking, sequences which hybridise under conditions of high stringency are included within the scope of the invention, as are sequences which, but for the degeneracy of the code, would hybridise under high stringency conditions.

A further aspect of the present invention provides an expression vector capable of expressing Fe effector peptides, preferably the Fc receptor binding effector peptides as described above. Preferably, the expression vectors comprise a nucleic acid molecule encoding the Fc effector peptides, particularly the Fc receptor binding effector peptides as described above. Alternatively, said expression vectors comprise a nucleic acid molecule encoding one or more polypeptides which form all or part of a binding site capable of binding a target molecule, together with nucleic acid sequences encoding said Fc effector peptides. Thus, said expression vectors can encode the Fc effector peptide alone or together with one or more polypeptides which form all or part of a binding site capable of binding a target molecule. Examples of possible types and structures of expression vectors according to this aspect are described below.

A yet further aspect of the present invention provides a host cell expressing an Fc effector peptide, particularly an Fc receptor binding effector peptide of the invention. Again, as with the expression vectors described above, said host cells may express the Fc effector peptide alone or together with one or more polypeptides which form all or part of a binding site capable of binding a target molecule. Also included are host cells containing expression vectors of the invention as defined herein. Examples of possible host cells which may be used to express such Fc effector peptides are described below.

Finally, a yet further aspect of the present invention provides a method of producing an Fc effector peptide, particularly an Fc receptor binding effector peptide of the invention, comprising the steps of (i) growing a host cell containing a nucleic acid molecule encoding an Fc effector peptide of the invention under conditions suitable for the expression of the Fc effector peptide; and (ii) isolating the Fc effector peptide from the host cell or from the growth medium/supernatent. Alternatively, the Fc receptor binding effector peptides may be produced by direct peptide synthesis using methods well known and documented in the art.

Appropriate Fc effector peptides for use in the binding molecules of the present invention, which display one or more effector functions associated with the constant region (Fc) of an immunogloblin heavy chain can be identified by any suitable technique. For example, soluble complement proteins, Fc receptors, or cells expressing Fc receptors can be used as targets in affinity selection or screening procedures to isolate novel Fc effector peptides from combinatorial libraries. Such peptides, selected for binding immunoglobulin effector ligands, have potentials to activate natural immunoglobulin effector functions. Libraries of peptides displayed on filamentous phages exemplify one such source of novel peptides.

After identification of candidate Fc effector peptides by appropriate methods, e.g. using phage display, the assessment of the selected peptides for appropriate Fc effector activities can be carried out by appropriate methods which would be routine to a person skilled in the art.

For example in the case of peptides which are candidates to induce complement activation this can be readily assayed by evaluating the ability of peptides to bind a component of the complement pathway, e.g. the C1q protein of the C1 protein complex. In addition, appropriate test systems to assay the ability of the peptides to induce production of other proteins in the complement cascade, such as C3b protein can also be designed, as can test systems to assay peptides which can trigger complement dependent lysis of cells, by for example measuring the release of ⁵¹Cr from target cells in the presence of complement proteins, e.g. in the form of serum.

In the case of Fc receptor binding, the ability of the candidate peptides to bind purified Fc receptors or to cells expressing the particular Fc receptor can be assessed. In addition, the ability of candidate peptides to trigger cell mediated destruction of target cells may be assessed by measuring ⁵¹Cr release from target cells in the presence of suitable cytotoxic cells, e.g. mononuclear cells, macrophages, etc. The ability of candidate peptides to mediate trancytosis of molecules from the basolateral to the apical surface of epithelium can be assessed using an in vitro epithelial cell system such as the MDCK system described in the Examples herein.

Thus a yet further aspect of the invention provides a method of producing an Fc effector peptide displaying one or more effector functions associated with the constant region (Fc) of an immunoglobulin heavy chain and preferably an Fc effector peptide which has the ability to bind one or more Fc-receptors, for use in the binding molecules of the present invention, comprising the step of screening a library of candidate peptides to select one or more Fc effector peptides which display the appropriate Fc effector function and further comprising manufacturing one or more of said selected peptides or a derivative thereof and optionally formulating said peptide or derivative with at least one pharmaceutical carrier or excipient.

Said “derivatives” include peptides which have been modified in any appropriate way but still retain the Fc effector function selected for. Indeed, the Fc effector function may well be improved by such derivatization. Thus, said derivatives include peptides which are “substantially homologous” to the selected peptides according to the definition provided herein, i.e. also including functionally equivalent variants and related sequences as defined herein. Said derivatives can be produced by modifying the selected Fc effector peptides or can be resynthesised, e.g. produced by direct peptide synthesis, using methods well known and documented in the art. Similarly the manufacturing step may involve the resynthesis of selected peptides using the sequence information derived from the screening step.

The above methods may further comprise the optional step of incorporating said selected peptide or a derivative thereof into a binding molecule of the invention before the manufacturing step thereby manufacturing a binding molecule rather than an effector peptide which can then optionally be formulated with at least one pharmaceutical carrier or excipient.

The binding molecules of the invention may be prepared using techniques which are standard or conventional in the art. Generally these will be based on genetic engineering techniques which will allow expression of the binding molecule, or a part thereof, in the form of a fusion protein, but protein manipulation techniques or proteolytic digestion to release a selected domain, polypeptide or peptide and chemical coupling of the binding site polypeptide(s) with the Fc effector peptides is also possible, using known techniques.

Generally, in the techniques based on genetic engineering, a genetic construct is prepared which comprises nucleic acid sequences which encode the various binding site polypeptides and Fc effector peptides of the desired recombinant binding molecule. Appropriate nucleic acid sequences encoding the various binding site polypeptides can be derived from any appropriate source. In preferred embodiments the binding site is an antibody fragment, for example a Fab fragment, scFv fragment or Fv fragment. Appropriate sources of the various antibody domains making up these fragments, e.g. V_{H}, V_{L}, C_{H1} and C_{L} would be well known to a person skilled in the art as would the appropriate content and arrangement of genetic constructs to produce the particular antibody fragment chosen. For example, the various domains can be derived from one or more naturally occurring antibody genes or may be sequences which are substantially homologous thereto or variants thereof which may be produced for example by one or more of single or multiple base addition, deletion or substitution. Alternatively, said antibody domains may be wholly or partially synthetic, e.g. may not correspond or derive from naturally occurring antibody immunoglobulin genes but comprise one or more random or semi random nucleotide sequences.

In general, where a scFv fragment is to be encoded appropriate nucleic acid sequences encoding-suitable V_{H} and V_{L} domains making up the antigen binding site would be obtained from appropriate sources and connected in a genetic construct by a sequence encoding a peptide linker. The design of the linker would again be well within the bounds of a skilled person, the major purpose being to allow the heavy and light chains to be sufficiently spaced apart so that they can interact to adopt an appropriate conformation to enable an antigen binding site to be formed.

For a Fab fragment or an Fv fragment on the other hand, rather than all the domains making up the antigen binding site being present in the same single nucleic acid construct and expressed as one polypeptide molecule, the appropriate components of the heavy and light chains of the antibody fragment (i.e. V_{L} and V_{H} in the case of Fv fragments and V_{L}, C_{L} and V_{H}, C_{H}1 in the case of Fab fragments) are generally expressed as separate molecules/polypeptide chains which then associate together within the host cell to form the antigen binding site and are secreted. In this case two separate genetic constructs can be designed. Alternatively, the separate polypeptide chains can be encoded by nucleic acid sequences on the same construct but with appropriate control sequences arranged so as to ensure that the polypeptide chains are expressed as separate molecules which can then associate in the host cell, rather than being expressed connected by a linker as in the case of scFv.

The Fc effector peptide is generally associated with one or more of the binding site polypeptides by producing it as a fusion protein, i.e. a nucleic acid sequence encoding the Fc effector peptide is incorporated in a genetic construct such as those described above in such a position that the Fc effector peptide is expressed in the same molecule i.e. as part of the same polypeptide as at least one of the polypeptide domains making up the binding site. In all cases, the position of the nucleic acid encoding the Fc effector peptide in the construct is chosen such that, when expressed, the binding site and the Fc effector peptide are functional. The appropriate design of the genetic constructs to achieve this would be routine practice to someone skilled in the art. Preferably the nucleic acid encoding the Fc effector peptide is positioned C-terminally of the nucleic acid encoding the polypeptide domain making up all or part of the binding site.

Thus in the case of constructs where the polypeptides making up the binding site are produced as a single polypeptide chain, e.g. scFv antibody fragments, the nucleic acid fragment encoding the Fc effector polypeptide is incorporated within the construct so that the Fc effector polypeptide is produced on the same polypeptide chain as the V_{H} and V_{L} domains. In the case of constructs where the polypeptides making up the binding site are produced as more than one separate chain, e.g. Fab fragments or Fv fragments then the Fc effector polypeptide can be incorporated into the genetic construct so that it is formed as a fusion protein with any one or all of the polypeptide chains which will subsequently associated together to form the binding site.

If the Fc effector peptide part of the binding molecule is not produced as a fusion protein, it can be generated by methods of direct peptide synthesis and subsequently associated with or attached to the polypeptides making up the binding site by any appropriate molecular or chemical linkage.

The genetic constructs or vectors of the invention generally additionally contain other appropriate components or regulatory elements which enable the induction and regulation of expression of the polypeptides and peptides encoded by the construct in the particular host cell system chosen. Examples of appropriate components include appropriate control sequences such as for example transcriptional control elements (e.g. inducible or non-inducible promoters, enhancers, termination stop sequences) and translational control elements (e.g. start and stop codons, ribosomal binding sites) linked in matching reading frame with the nucleic acid molecule encoding the polypeptide desired to be expressed. Optional further components of such vectors include for example replication origins, selectable markers, antibiotic resistance genes, general tags or reporter molecules or secretion signalling and processing sequences.

The genetic constructs are generally expressed by standard techniques involving the introduction of one or more nucleic acid constructs as described above into a host cell and the expression of the polypeptide or polypeptides therefrom. Generally, if the components making up the binding site are encoded in two different genetic constructs, these should be co-introduced into the same host cells to enable co-expression and association of the polypeptides to occur before they are secreted by the host cell.

Any appropriate eukaryotic or prokaryotic host cell can be used for expression, including bacterial (e.g. E. coli), baculovirus, yeast, fungal, insect, plant or mammalian cells, but particularly preferred host cell systems are bacterial systems such as E. coli. The expression of small antibody fragments in bacterial cells such as E. coli is described for example in Kipriyanov et al., J. Immunol Methods; 1997, 200: 69-77 and similar expression methods can be used to express the binding molecules or the effector peptides of the present invention. Generally speaking, those skilled in the art are well able to construct vectors and design protocols for the expression of recombinant proteins and more detail in this regard will not be provided herein.

As described above, nucleic acid molecules comprising nucleic acid sequences which encode one or more polypeptides which form all or part of a binding site capable of binding a target molecule, together with nucleic acid sequences which encode one or more Fc effector peptides displaying one or more effector functions associated with the constant region (Fc) of an immunoglobulin heavy chain form yet further aspects of the invention.

For example where the binding molecule contains an scFv antibody fragment as a binding site, the nucleic acid molecules of the invention may comprise sequences encoding a V_{H} polypeptide and a V_{L} polypeptide separated by a sequence encoding a peptide linker. Such nucleic acid molecules will also comprise one or more sequences encoding one or more Fc effector peptides.

Alternatively in embodiments where the binding site of the binding molecule is a multichain polypeptide, i.e. the binding site is formed from more than one polypeptide (e.g. the binding site is an Fv antibody fragment or a Fab antibody fragment or some other non-antibody multi-chain binding site, e.g. derived from a multi-chain receptor molecule), then the nucleic acid molecules of the invention will comprise sequences encoding at least one of the polypeptide chains making up the multi-chain binding site together with one or more sequences encoding an Fc effector peptide. For example, in the case where the binding molecule is an Fv fragment, the nucleic acid molecules may comprise a sequence encoding a V_{H} polypeptide and one or more sequences encoding one or more Fc effector peptides, optionally together with a sequence encoding a V_{L} polypeptide. Alternatively, the nucleic acid molecules may comprise a sequence encoding a V_{L} polypeptide and one or more sequences encoding one or more Fc effector peptides, optionally together with a sequence encoding a V_{H} polypeptide. In the case where the binding molecule is a Fab antibody fragment, the nucleic acid molecules may comprise a sequence encoding a V_{H} polypeptide, a sequence encoding a C_{H}1 polypeptide and one or more sequences encoding one or more Fc effector peptides. Optionally, such nucleic acid molecules may also comprise a sequence encoding a V_{L} polypeptide and a sequence encoding a C_{L} polypeptide. Alternatively the nucleic acid molecules may comprise a sequence encoding a V_{L} polypeptide, a sequence encoding a C_{L} polypeptide and one or more sequences encoding one or more Fc effector peptides. Optionally such nucleic acid molecules may also comprise a sequence encoding a V_{H} polypeptide and a sequence encoding a C_{H}1 polypeptide.

The nucleic acid molecules according to the present invention may include cDNA, RNA, genomic DNA (including introns) and modified nucleic acids or nucleic acid analogs (e.g. peptide nucleic acid). The nucleic acid molecules may be wholly or partially synthetic. In particular they may be recombinant in that nucleic acid sequences which are not found together in nature (i.e do not run contiguously in nature) have been ligated or otherwise combined artificially. Alternatively, they may have been synthesised directly e.g. using an automated synthesiser. Thus, as described elsewhere herein the sequences making up the nucleic acid molecules may be derived from or comprise naturally occurring antibody genes or variants thereof or wholly or partially synthetic sequences.

Expression vectors comprising the nucleic acid molecules of the invention as defined above form yet further aspects of the invention, as do host cells expressing the nucleic acid molecules of the invention.

Methods of producing the binding molecules of the invention comprising the steps of (i) the expression in a host cell of a nucleic acid molecule encoding one or more polypeptides which form all or part of a binding site capable of binding a target molecule and one or more Fc effector peptides and (ii) the isolation of the expressed binding molecules from the host cells or from the supernatent/growth medium form a yet further aspect of the invention.

As described above, in embodiments where the binding site is made up of more than one polypeptide, the other polypeptides are preferably also expressed in the host cell, either from the same or a different expression vector, so that the complete binding molecules can assemble in the host cell and be isolated therefrom.

The binding molecules of the invention have a defined specificity due to the polypeptide(s) making up the binding site which are capable of specifically binding a target molecule. For example, the binding site may comprise an antibody fragment or be derived from a receptor, hormone, antigen, enzyme or other ligand. Thus, the binding site can be used to target the binding molecules of the invention to for example particular body sites or cell types or foreign microorganisms, whereupon the particular Fc effector function or functions conferred by the Fc effector peptides can act on the target site, organism, or cells. Thus, it is envisaged that the binding molecules of the invention can be used to treat any disease where the stimulation of immunoglobulin effector function is useful.

Where the binding molecules are pepbodies, these mimic intact immunoglobulins and thus can be used as antibody therapeutics in any disease where Fc effector function such as binding Fc receptors or the activation of complement is advantageous.

Due to the specificity of targeting of the binding molecules of the present invention, these binding molecules can also be used for the imaging of body sites if an appropriate label is attached. Such binding molecules can also be used in in vitro or in vivo diagnosis of disease.

Because the Fc effector peptides induce a response based on that which would be induced by natural intact immunoglobulins in the body (e.g. utilise the body's own elimination system for target destruction), therapy using the binding molecules of the present invention is likely to be a more effective form of therapy and less immunogenic than for example the targeting of other therapeutic fusion proteins, e.g. the targeting of a fusion protein containing a foreign cytotoxic agent to a cellular target. However, if desired, the binding molecules of the invention can be used to target and deliver additional drugs or compounds, such as cytotoxic or beneficial drugs or compounds to a particular target site or entity by attaching or conjugating etc., such compounds or drugs to the binding molecules by any appropriate means.

In all the therapeutic uses the small size of the binding molecules is a distinct advantage as this facilitates a more rapid and efficient penetration of body tissues. In particular in the preferred embodiments of the invention where the binding site is an antibody fragment (i.e. the binding molecules are pepbodies), the reduction in size compared to intact antibodies is very significant (see Table A).

TABLE A

| Protein | Approximate MW | Glycosylated |
|---|---|---|
| IgG | 150 | Yes |
| ScFv pepbody | 30 | No |
| ScFv2 pepbody | 60 | No |
| Fab pepbody | 50 | No |
| Fab2 pepbody | 100 | No |

(2 = two antigen binding sites)

In addition, as mentioned above the binding molecules of the invention and in particular the pepbodies do not require glycosylation for function (see Table A). This is not only advantageous in terms of production (i.e. they can be produced in prokaryotic hosts) but also means that they are less likely to be recognised and rejected by the host immune system. Thus, the pepbodies and other types of binding molecule are therapeutic reagents based on the body's own immune system.

As discussed above, the main Fc effector functions which can be induced by the Fc effector peptides are interaction with Fc receptors and complement activation. Complement activation triggers an immune cascade and the recruitment of a number of cells involved in the immune response, e.g. neutrophils, eosinophils, monocytes, macrophages and B cells. In addition certain Fc receptors are located on immune effector cells such as monocytes, macrophages, neutrophils, eosinophils, etc. Thus, it can be seen that the targeting of the binding molecules of the invention, which contain appropriate Fc effector peptides to induce complement activation and/or recruit phagocytes or other cellular components of the immune system via interaction with Fc receptors on these cells, to a specific target can result in a relatively local immune response to that target and the subsequent disruption, damage, ingestion or preferably elimination of the target entity in question. Thus, preferred targets to which the binding sites of the binding molecules are directed are those that it is wished to damage or eliminate, e.g. tumour cells or other unwanted foreign bodies or microorganisms such as viruses, bacteria etc.

Where the Fc effector peptide has the ability to bind to the FcRn receptor, binding molecules comprising such peptides can cross the placenta into the neonate. Such binding molecules are therefore targeted to the neonate and can be used for example in the treatment, for example the prophylactic treatment of the neonate. Furthermore, another important aspect with the Fc effector peptides which have the ability to bind the FcRn receptor is that this receptor also mediates the retention of antibodies in intracellular vesicles in endothelial cells lining blood vessels in vivo. Thus binding molecules comprising FcRn-binding peptides and in particular pepbodies comprising FcRn binding peptides will remain in the body circulation longer than normal antibody fragments. This increased serum half life is an important advantage, as one of the main issues with regard to the general use of antibody fragments as therapeutic agents is the fact that the current antibody fragments are too unstable and they do not remain in the circulation long enough to induce their therapeutic effects. Such problems are likely to be much improved with the binding molecules of the present invention which comprise FcRn binding effector peptides, as these will stay in the body long enough to exert a therapeutic effect. Thus, such binding molecules which display an increased serum half life, or a general increased stability compared to binding molecules, and in particular antibody fragments, which do not contain such effector peptides, form a yet further preferred aspect of the invention.

Finally, where the Fc effector peptide has the ability to bind to the pIgR, binding molecules comprising such peptides can be delivered to mucous membranes of epithelial cells. Since the ability to adhere to the epithelial cells of mucous membranes is an essential step in the mechanism by which many foreign organisms (e.g. viruses, bacteria, fungi, etc.) enter the body, being able to target binding molecules of the invention to these mucosal surfaces will be useful in combatting, controlling or alleviating infection or disease. This is especially the case as, by appropriate selection of the binding site component, the binding molecules can be designed to specifically bind, coat or attack the foreign organisms present at the mucosal surfaces, thereby reducing or preventing their infection of the mucosal epithelium.

Thus, it can be seen that a yet further aspect of the invention provides the binding molecules or the Fc effector peptides as defined herein for use in therapy, diagnosis or imaging.

A yet further aspect of the invention provides the use of the binding molecules or the Fc effector peptides as defined herein in the manufacture of a composition or medicament for use in therapy, imaging or diagnosis.

Methods of treatment of a subject comprising the administration of an appropriate amount of a binding molecule as defined herein to a subject, or to a sample (e.g. a blood sample) removed from a subject and which is subsequently returned to the subject, provide yet further aspects of the invention.

If the Fc effector peptides are used in the above described uses and methods then these may be administered locally at the site where action is required or may be attached or othewise associated with entities which will facilitate the targeting of the Fc effector peptides to an appropriate location in the body.

Yet further aspects are methods of diagnosis or imaging of a subject comprising the administration of an appropriate amount of a binding molecule as defined herein to the subject and detecting the presence and/or amount of the binding molecule in the subject.

Appropriate diseases to be treated in accordance with the above described uses and methods include any disease where the stimulation of Fc effector function, such as binding Fc receptors (and the subsequent biological effects induced thereby) or the activation of complement is advantageous. Examples of such disease include cancer and any diseases involving the presence in the body of foreign organisms or foreign proteins or is antigens, e.g. viral, fungal or bacterial infections.

The terms “therapy” or “treatment” as used herein include prophylactic therapy. In particular, in embodiments where the Fc effector peptides can bind to the FcRn receptor, binding molecules comprising such peptides can cross the placenta into the neonate. Thus, in this way the binding molecules of the invention can be used for the treatment, e.g. the prophylactic treatment, of neonates. For example, where the binding molecules are pepbodies, the present invention provides a way of introducing into the neonate protective or otherwise useful antibodies which will not be regarded as foreign. The terms “therapy” and “treatment” include combatting or cure of disease or infections but also include the controlling or alleviation of disease or infection or the symptoms associated therewith.

Pharmaceutical compositions comprising the binding molecules or the Fc effector peptides as defined herein, together with one or more pharmaceutically acceptable carriers or excipients form a yet further aspect of the invention.

The binding molecules or the Fc effector peptides as defined herein may also be used as molecular tools for in vitro applications and assays. As the binding molecules can still function as members of specific binding pairs then these molecules can be used in any assay where the particular binding pair member is required. For example, in the embodiments when the binding molecules are pepbodies which can bind particular antigens these molecules can be used in any assay requiring an antibody with a specificity for that particular antigen.

Thus, yet further aspects of the invention provide a reagent which comprises a binding molecule or an Fc effector peptide as defined herein and the uses of binding molecules or Fc effector peptides as defined herein to induce one or more types of Fc effector activity, such as the binding to Fc receptors or the activation of complement. Kits comprising a binding molecule or an Fc effector peptide as defined herein form a yet further aspect.

By using the binding molecules as molecular tools, in vitro diagnosis could also be carried out on a sample of fluid, tissue etc. derived from a subject. Thus, methods of in vitro diagnosis involving the use of binding molecules or Fc effector peptides as defined herein form a yet further aspect of the invention.

The invention will now be further described by way of the following non-limiting examples with reference to the following Figures in which:

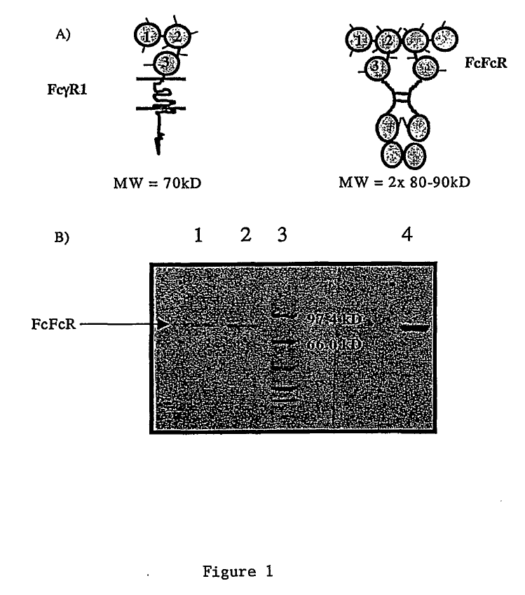

FIG. 1A) shows a schematic drawing of FcγRI and FcFcR, the extracelluar part of the human FcγRI genetically fused to human IgG4. FIG. 1B) shows an SDS PAGE of FcFcR produced in methionin pulsed NSO cells immunoprecipitated with both anti-FcR (lane 1) and anti-Fc antibodies (lane 2). Lane 3: C¹⁴ labelled molecular weight standard. Lane 4: FcFcR in NSO cell lysate detected by HRP conjugated protein A in Western blot. The FcFcR molecule migrates as an 80 kDa band corresponding to the monomer fraction.

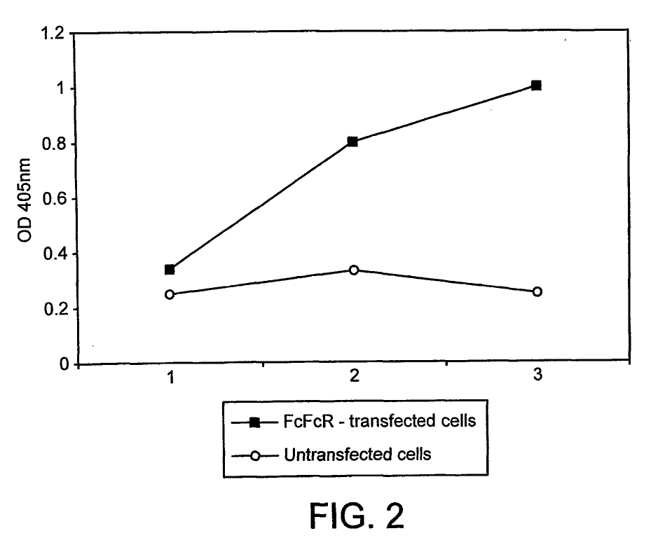

FIG. 2 shows IgG binding activity of NSO lysate. Lysate from FcFcR transfected and untransfected NSO cells were added to protein A coated wells. To increase the amount of immobilised FcFcR, lysate was added up to 3 times. Biotinylated human IgG3, streptavidin and HRP conjugated biotin was added. Absorbance at 405 nm was measured 1 h after addition of substrate (ABTS).

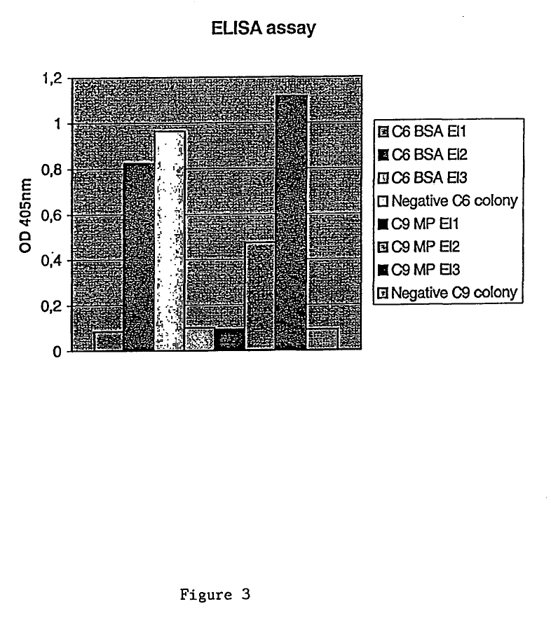

FIG. 3 shows portions of amplified eluates (El1-3) from three rounds of affinity selection which were measured for SC-binding in an ELISA assay. Both the second and third round from the C6- and C9-libraries gave at least 4 times higher signal than the first round and the negative control. The C6-library was blocked with 1% BSA, whereas the C9-library was blocked in 1% milk powder (MP).

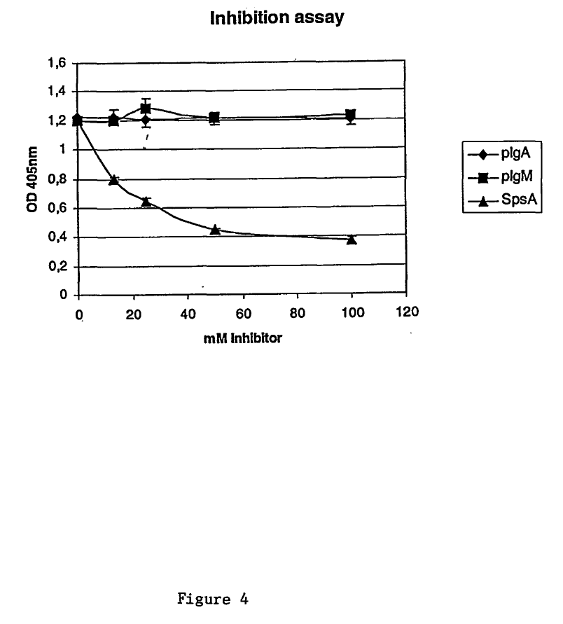

FIG. 4 shows that SpsA (streptococcus pneumoniae secretory IgA binding protein which binds SC) but not IgA or IgM competed with two different phage particles displaying SC binding peptides. Shown are the results for phages displaying the peptide CWTSGARWRLC.

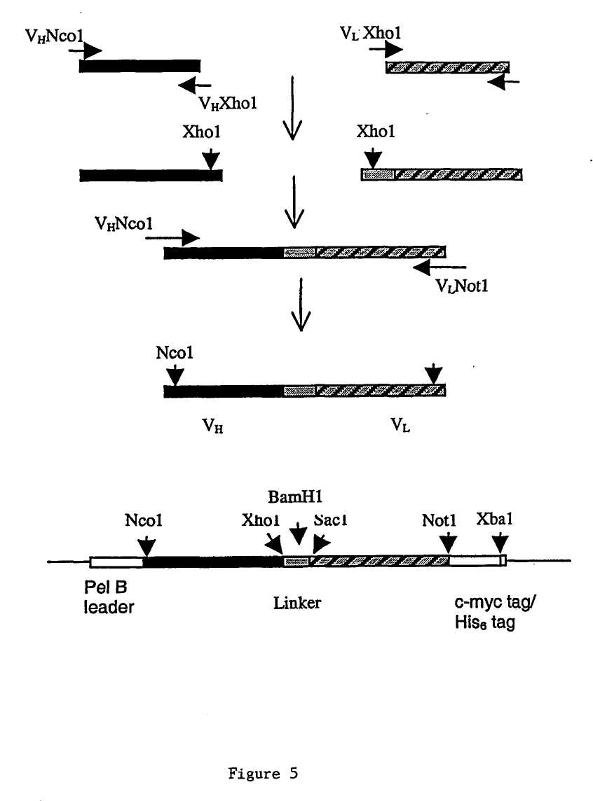

FIG. 5 shows the generation of anti NP/Nip ScFv in the bacterial expression vector pHOG.

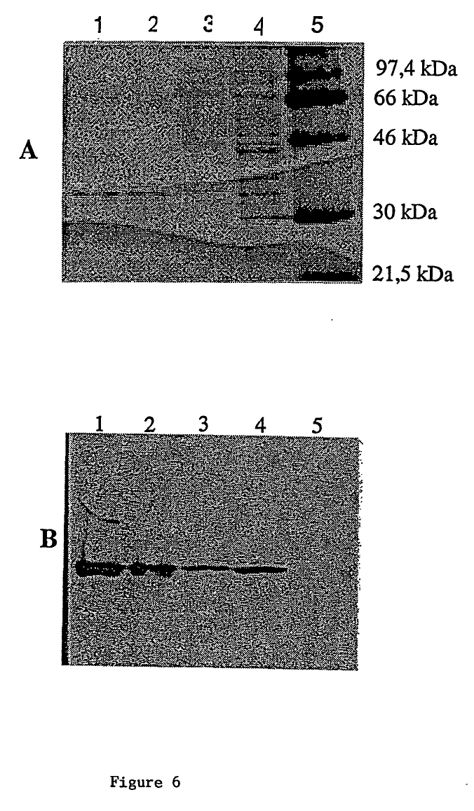

FIG. 6 shows the expression and purification of ScFv with Nip/NP specificity. Panel A) shows a 10% SDS-PAGE stained with Coomassie Blue. Lanes 1 and 2: Affinity purified ScFv from the growth medium; lane 3: Growth medium; lane 4: periplasmic content; lane 5: Molecular mass marker. Panel B) shows a Western blot of the same gel as in A. The antibody fragments were detected with biotinylated goat anti mouse λ-light chain (SOUTHERN BIOTECHNOLOGY ASSOCIATES, INC), HRP conjugated streptavidin and ECL solutions (Pharmacia Amersham).

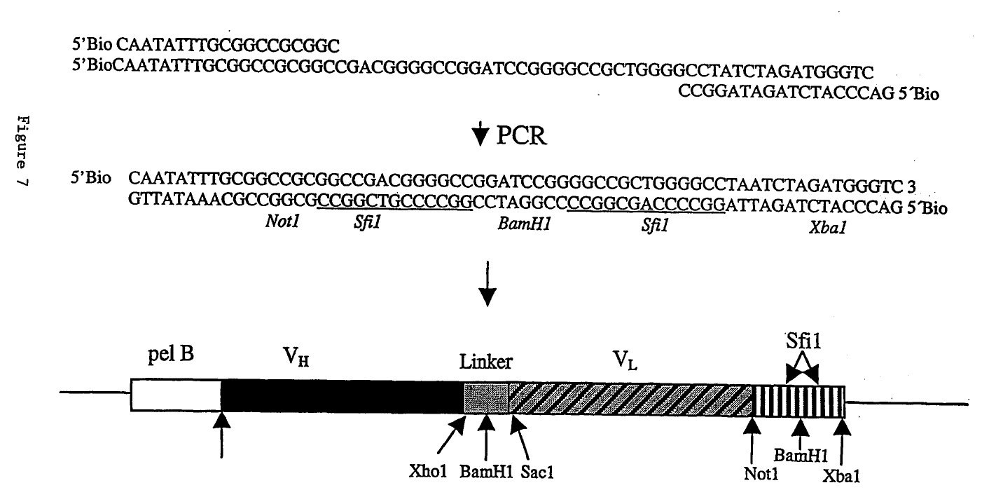

FIG. 7 shows the vector pSG1A. The c-myc/His6 tag of pHOG antiNP/Nip was exchanged for a small DNA insert containing fUSE5 compatible sfiI sites.

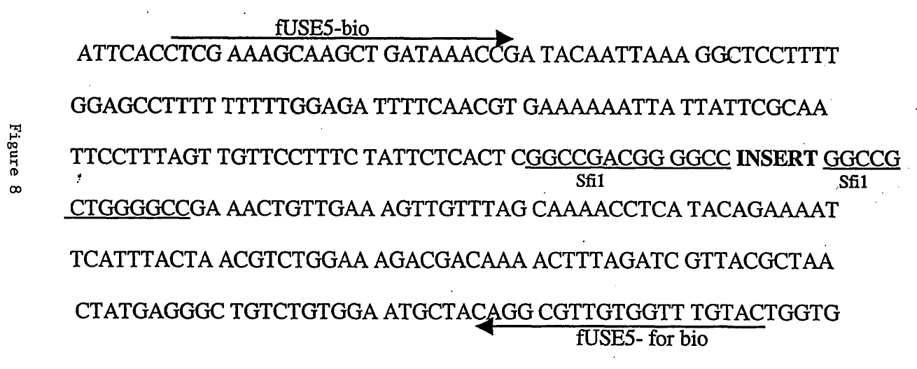

FIG. 8 shows a schematic for the PCR amplification of peptide-encoding inserts from fUSE5 phage display libraries. Horizontal arrows indicate fUSE5 primers fUSE5-bio and fUSE5-for-bio annealing 132 bp upstream and 142 bp downstream of the cloning site containing the sequence encoding a displayed fusion product.

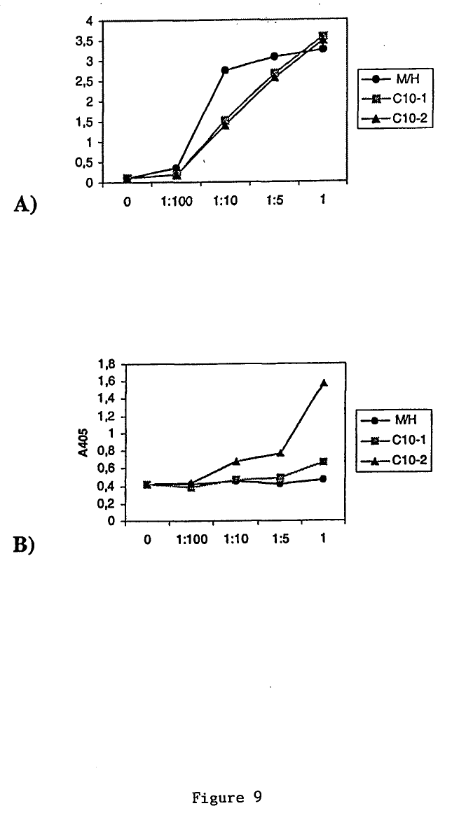

FIG. 9 shows ELISA assays demonstrating antigen (Nip) and C1q binding. Opical density at 405 nm was measured 1 hour after addition of substrate. Panel A) shows binding of dilutions of antibody fragments to the hapten Nip. M/H indicates fragments with a c-myc/His6 tag, C-10-1 and C10-2 indicates fragments with C-terminal C1q binding peptides. Panel B) shows binding of C1q to the immobilised antibody fragments.

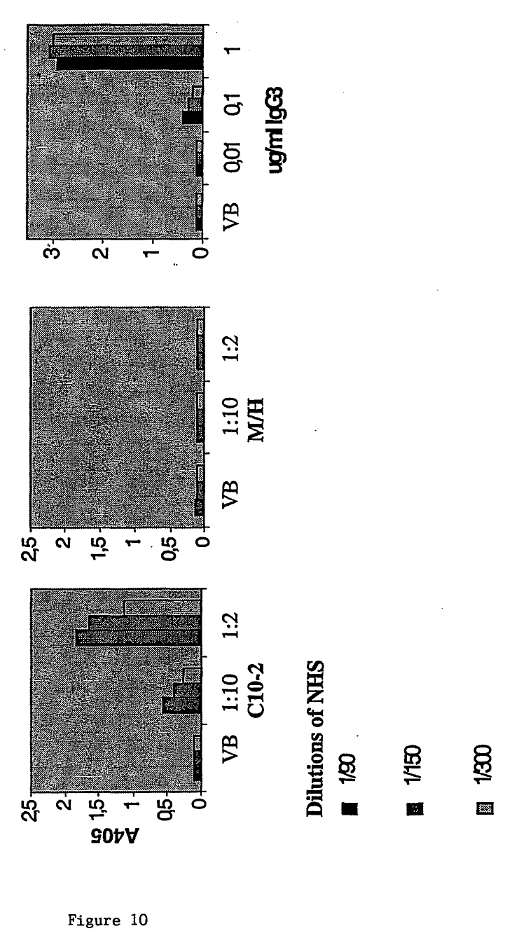

FIG. 10 shows complement fixation/C1q binding from serum. VB veronal buffer; Supernatants from antibody fragment producing cells were diluted 1:10 and 1:2 with VB. IgG3 was diluted as indicated. The C1q binding in different dilutions of NHS was measured by a sandwich ELISA using rabbit anti human C1q, HRP conjugated sheep anti rabbit serum and ABTS. Signals are read 1 h after addition of ABTS.

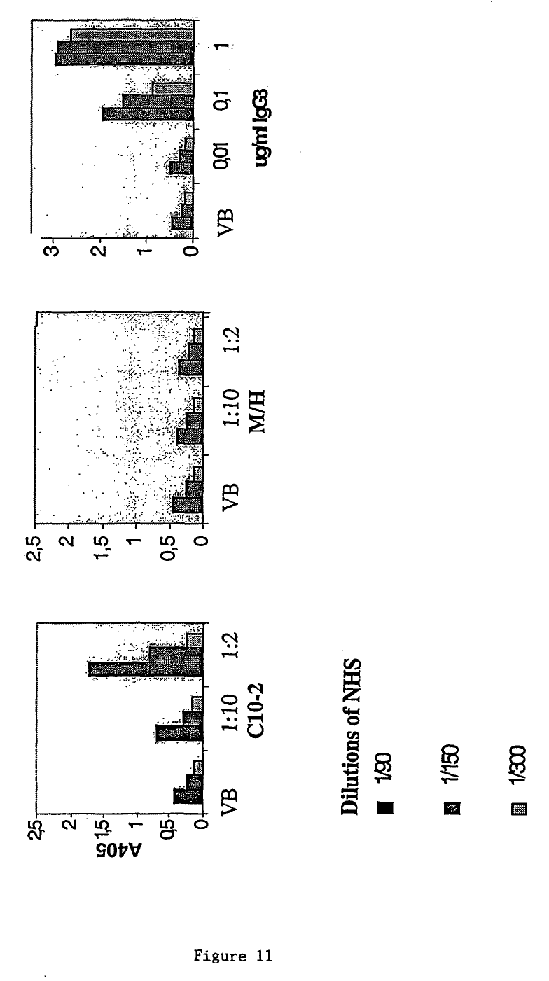

FIG. 11 shows complement activation/C3 deposition from serum. VB veronal buffer; Supernatants from antibody fragment producing cells were diluted 1:10 and 1:2 with VB. IgG3 was diluted as indicated. The C3 deposition from different dilutions of NHS was measured by a sandwich ELISA using rabbit anti human C3, HRP conjugated sheep anti rabbit serum and ABTS. Signals are read 1 h after addition of ABTS.

FIG. 12 shows a schematic of an intact immunoglubulin. The Fv, Fab and Fc regions are highlighted.

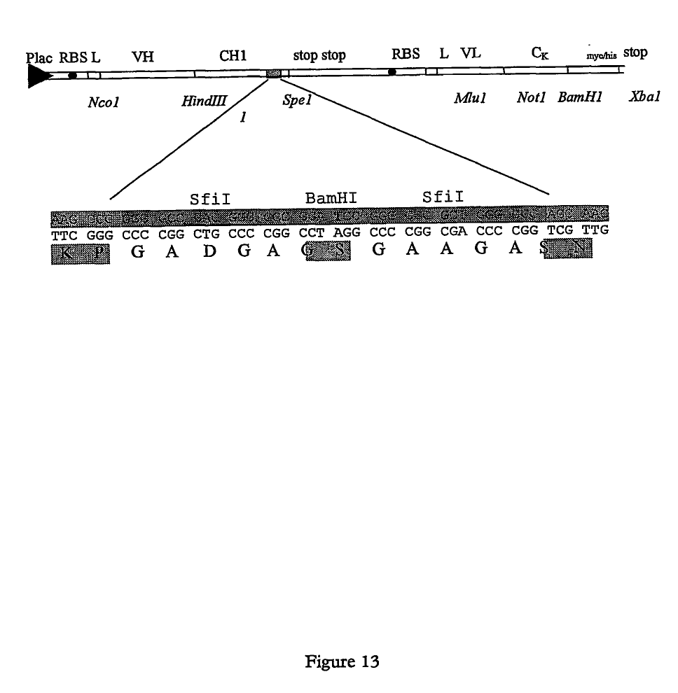

FIG. 13 shows the structure of the plasmid pFab SfiIL6 which encodes an anti pHOx Fab and into which a C1q binding peptide is inserted in the f-g loop (loop 6, L6) of the Fab fragment.

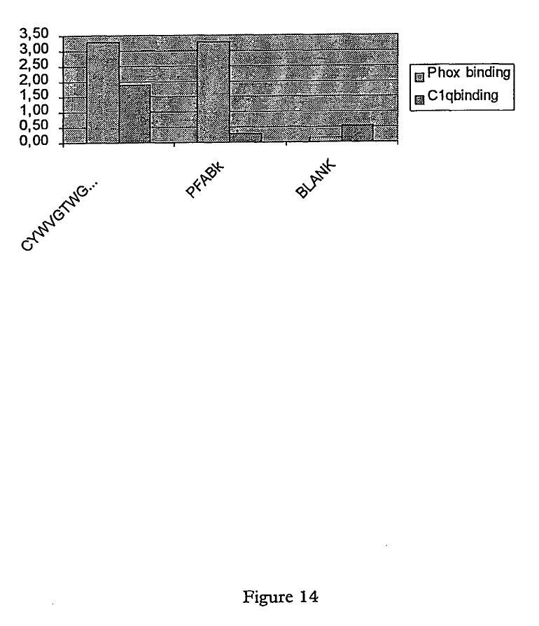

FIG. 14 shows ELISA assays demonstrating antigen (Phox) and C1q binding to Fab fragments which contain a C1q binding peptide (CYWVGTWG . . . ) or do not contain such a peptide (PFABK). Binding to blank plates is also shown as a control.

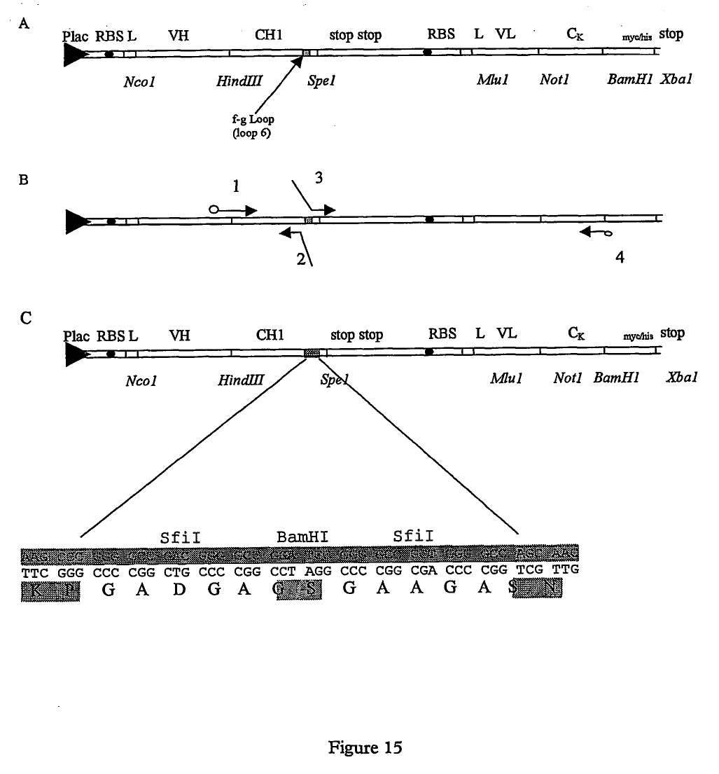

FIG. 15 shows the construction of the plasmid pFab SfiIL6 which encodes an anti pHOx Fab vector. Panel A shows the original Fab plasmid. Panel B shows PCR SOEing (Splicing by Overlap Extension) by the four oligonucleotides

| | |
|---|---|
| 1: | 5′ Bio CATCCGCCCCAAAGCTTGCCTCCACC 3′, |
| 2: | 5′ GGCCCCAGCGGCCCCGGATCCGGCCCCGTCGGCCCCGGGCTTGT |
| | GATTCACGTTGCAGATG 3′, |
| 3: | 5′ GGGGCCGACGGGGCCGGATCCGGGGCCGCTGGGGCCAGCAACAC |
| | CAAGGTGGACAAGAAAG 3′ and |
| 4: | 5′ Bio TATAATAGGATCCCCCACAGTCTCCCCTGTTGAAGCT 3′ |

Panel C Shows the final plasmid construct pFab SfiI L6 which encodes an anti phOx Fab. Into the SfiI sites DNA encoding different peptides can be inserted.

### EXAMPLES

### Example 1

Identification of Fc-γ Receptor Binding Peptides

The human monocytic cell line U937 constitutively expresses both FcγRI and FcγRII (van de Winkel and Anderson, J Leukoc Biol. 1991 May; 49(5):51-1-24.). Stimulation with INF-γ induces the expression of an increased number of FcγRI (Guyre et al. J Clin Invests. 1983 July; 72(1):393-7). This example demonstrates how phage displayed peptides can be selected for binding INF-γ stimulated U937 cells and that these peptides can also bind to a recombinant soluble form of FcγRI.

Library construction: The fUSE5 vector was used to generate two libraries of cysteine constrained peptides displayed as fusions to phage protein III (essentially as described in Smith and Scott Methods Enzymol. 1993;217:228-57). The peptides had the length of six (C6-library) and nine (C9-library) random amino acids, between two invariable cysteines.

Based on the number of primary transformants with a productive insert (>90%) the libraries were estimated to consist of 5×10⁷ (Cys6) and 1×10⁸ (Cys9) different clones.

Cells: U937 cells (ATCC CRL-1593) and K562 cells (ATCC CCL-243) were maintained in RPMI 1640 medium (Gibco laboratories) supplemented with 10% heat-inactivated FCS (Biological Industries, Beth Haemark), 2 mM L-glutamine, 100 U/ml penicillin (Gibco). Cells-were incubated at 5% CO₂ and 37° C. at density ranging from 10⁵ to 10⁶ cells/ml.

The monocytic cell line U937 constitutively expresses FcγRI and FcγRII at levels of approximately 10 000/cell and 50 000/cell respectively. FcγRI is readily upregulated by IFN-γ stimulation. U937 do not express FcγRIII.

To increase FcγRI expression the U937-cells were treated with 100 U/ml IFN-γ for 40 hours.

Affinity selection: For affinity selection, 107 stimulated U937cells were washed in 20 ml pan-wash buffer (PBS pH7.4, 1% BSA, 1 mMCaCl₂, 10 mMMgCl₂) and then resuspended in 1 ml pan-wash buffer. Portions (1×10¹⁰ E. coli K91K transducing units (TU)) of each library were added to the cells. Cells and phages were incubated with agitation for 1.5 hours at 4° C. Unbound phages were removed by washing the cells 6 times with 2 ml pan-wash buffer. Bound phages were eluted in 200 μl 0.1M HCl-glycine pH 2.2 for 10 minutes on ice. The eluates were neutralised with 17 μl 1.5M Tris pH 8.8. Phages were amplified (essentially as described in Smith and Scott Methods Enzymol. 1993;217:228-57). A second round of affinity selection was performed using an input of 10⁹ phages.

The output of phage increased from the first to the second round. The output from the C6-library increased from 6×10⁻⁶% to 10⁻³%, while the output from the C9-library increased from 8×10⁻⁵% to 2×10⁻³%.

Single colonies of phage producing bacterial cells from the second round of selection were picked and expanded for further study.

Peptide sequences of affinity selected phage clones: PCR products covering the peptide coding insert were created using the primers fUSE5 for (5′ GTACAAACCACAACGCCTGTAG 3′) and fUSE5 (5′TCGAAAGCAACGTGATAAACC 3′). The PCR products were sequenced (GATC/GmBh Germany) using the primer (5′CCCTCATAGTTAGCGTAACG 3′) and the results shown in Table 1.

TABLE 1
Sequences of peptides displayed by C6 phages following
two rounds of panning on INF-γ stimulated U937 cells.

| | | | | | | | | | |
|---|---|---|---|---|---|---|---|---|---|
| C | L | R | S | G | R | G | C | (4*) | |
| C | L | R | S | G | L | G | C | (4) | |
| C | L | R | S | G | A | G | C | (2*) | |
| C | L | R | S | G | S | G | C | | |
| C | L | R | S | G | R | A | C | | |
| C | L | R | S | G | A | N | C | (2) | |
| C | L | R | S | G | L | H | C | (2) | |
| C | R | R | S | G | Q | G | C | | |
| C | L | Y | G | D | E | L | C | | |
| C | F | P | V | G | R | A | T | | C |

16 out of 19 sequenced single clones shared the motif CLRSGXXC (X is more variable than the rest of the amino acids).
Number in parantheses indicate individual isolates of each sequence.
*indicates the presence of 2 different DNA sequences encoding the same amino acid sequence.

Inserts sharing the motif CLRSGXXC (X is variable and the cysteines are constant) was found in the majority of phages following the second round of panning the C6-library. A very different sequence CSWIPGVGLVC (cysteines are constant), dominated among clones from the C9-library, as shown in Table 2.

TABLE 2
Sequences of peptides displayed by C9 phages following
two rounds panning on INF-γ stimulated U937 cells.

| | | | | | | | | | | | |
|---|---|---|---|---|---|---|---|---|---|---|---|
| C | S | W | I | P | G | V | G | L | V | C | (6) |
| C | R | R | A | T | A | G | C | A | G | C | |
| C | R | S | M | V | M | L | R | V | R | C | |
| C | G | R | V | N | T | W | L | P | Q | C | |
| C | S | A | G | R | A | C | C | R | Y | C | |

A phage with the motif CSWIPGVGLVC dominated (6 out of ten sequenced single clones) among the enriched phages.

Cell binding of individual phages: Phage expressing the peptide CLRSGLGC were analysed for binding unstimulated U937 cells, IFN-γ stimulated U937 cells, as well as K562 cells. Both U937 and K562 are human monocyte cell lines. Whereas INF-γ stimulated U937 cells express an increased number of FcγRI, K562-cells preferentially express FcγRII. 5×10⁷ TU of individual phage clones were added to 3×10⁶ cells in pan-wash buffer, incubated and eluted as described above. The number of bound phages was determined as E. coli K91K-TU and the results shown in Table 3.

TABLE 3
Phage binding to cells from human monocytic cell lines.

| Cell-line | U937-INF-γ | | U937 | | K562 | |
|---|---|---|---|---|---|---|
| Phage clone | CLRSGLGC | CONTROL | CLRSGLGC | CONTROL | CLRSGLGC | CONTROL |
| Input | 5 × 10⁷ | 5 × 10⁷ | 5 × 10⁷ | 5 × 10⁷ | 5 × 10⁷ | 5 × 10⁷ |
| Output | 1 × 10⁵ | <2 × 10² | 1.4 × 10⁴ | <2 × 10² | 1.4 × 10⁴ | <2 × 10² |
| % output | 0.2% | <0.0004% | 0.028% | <0.0004% | 0.02% | <0.0004% |

The same amount of phage displaying the peptide CLRSGLGC, selected for binding INF-γ stimulated U937 cells, and a control phage displaying an irrelevant insert (CGPGGTVGYTC) were allowed to bind IFN-γ stimulated U937 cells, unstimulated U937 cells, as well as K562 cells. Unbound phages were removed by extensive washing. The number of bound phages was determined as TU's in acid eluates. The assays were repeated twice with similar results using new batches of cells.

More than 500 times more CLRSGLGC phages were eluted from IFN-γ stimulated U937cells compared to the irrelevant control phage (table 3). The CLRSGLGC phage bound significantly better to IFN-γ stimulated U937 cells compared to the same number of unstimulated U937 cells and K562 cells (table 3). Notably the diameter of K562 cells is approximately 1.8 times larger than U937 cells giving a larger binding surface.

Production of a dimeric soluble FcγRI:“FcFcR”. A recombinant human FcγRI as a fusion to human IgG4 Fc region was constructed yielding a dimeric soluble molecule (FcFcR, shown in FIG. 1.). The Fc part of human IgG4 only binds very weakly to FcγR1 allowing the production of dimeric molecules that can be detected by and directionally immobilised with protein A.

10⁷ fresh IFN-γ stimulated U937 cells were handled as described in Pharmacias mRNA extraction kit. Fresh mRNA was used as template for pd(T)18 priming, according to the manufacturers protocol (Pharmacia). The primary amplification of FcγRI extracellular domains (ED) (from bp 107 to 912 according to Allen and Seed (Nucleic Acids Res. 1988 Dec. 23;16(24):11824) giving a 807 bp fragment (269aa) cDNA, was obtained by PCR of 7 μl cDNA by using 40 pmol of each of the primers: FcRIBACK: 5-atctctttgcagcctccatgg-3′

FcRIFOR: 5′-atgaaaccagacaggagttgg-3′.

The primary 0.8 Kb PCR product was reamplified with primers

FcRIBACKhindIII: 5′-gagagagagaAAGCTT|atctctcttgcagcc3′ introducing a HindIII site and

FcRIFORbamhI/apaI: 5′-gagagagagaGGATCCGGGCCC|atgaaaccagacagg3′ introducing BamHI and ApaI sites. The PCR product was cloned in M13 mp18 and M13 mp 19. The presence of a correct insert was verified by sequencing. A third PCR, in which a splice-donor (sd) site was introduced between the last codon of sFcR (CAT,His) and the BamHI-site by the primers:

FcRI Back HindIII: 5′GAGAGAGAGA|AAGCTT|ATC TCT TTG CAG CC FcR-Bam/SD: 5′GAGAGAGAGA|GGATCC|ACTCACC/ATG AAA CCA GAC AGG (sd-sequence underlined) was performed. The HindIII/BamHI digested sFcRI was subcloned into the constant heavy chain gene of human IgG4 on HindIII-BglII sites, thereby substituting the IgG4 CH1 exon with cDNA encoding FcγRI ED. Sequencing verified a functional FcFcR construct which was further subcloned into the mammalian expression vector pSecTagB (Invitrogen) on HindIII/BamHI sites.

The FcFcR protein was expressed in the mouse myeloma cell line NSO. A protein of approximately 80 kD was immunoprecipitated from lysate of transfected cells (FIG. 1), corresponding to FcFcR half-molecules.

IgG3 Binding Activity of FcFcR.

Protein A was immobilised in wells at 10 μg/ml in a volume of 200 μl at 4° C. overnight (ON). The wells were blocked with 1% BSA in PBS for 1 h at room temperature (RT). Lysate from FcFcR-transfected and untransfected cells were added to the wells and incubated at RT for 2 h. The wells were washed and new lysate was added zero, one or two times following 2 h incubation at RT. The wells were washed 7 times with PBS/0.5% Tween 20. 200 μl biotinylated human IgG3 in PBS (1 μg/ml) was added to the wells. The wells were washed as above and binding of biotinylated IgG3 was detected with streptavidin and HRP conjugated biotin (FIG. 2).

Lysates from FcFcR transfected NSO cells reveal a clear IgG binding activity compared to lysate from untransfected cells, indicating the presence of a soluble and functional form of FcγRI in the lysate.

Binding of Phage Displayed Peptides to Recombinant Dimeric FcγRI:

Wells were coated as described above with lysate from FcFcR transfected NSO cells added three times. Phages displaying the peptide CLRSGLGC were added to the wells and incubated at RT for 1.5 h. The wells were washed 8 times with PBS/0.5% Tween 20. Bound phages were eluted by incubation with 200 μl 0.1M HCl-glycine pH 2.2 for 10 min. and neutralised with 17 μl 0.1M Tris pH 8.8. The number of bound phages were determined as E. coli K91K TU's (Table 4).

TABLE 4
Binding of phages to wells coated with
protein A and cell lysate from FcFcR transfected
NSO cells and untransfected NSO cells. Background
binding to protein A coated wells blocked with BSA
was 0.025%. The assays were repeated several times
revealing the same tendency.

| Cell-lysate source | FcFcR transfected cells | Untransfected cells |
|---|---|---|
| Phage clone | CLRSGLGC | CLRSGLGC |
| Input | 2.5 × 10⁸ | 2.5 × 10⁸ |
| Output | 6.8 × 10⁵ | 1.2 × 10⁵ |
| % output | 0.3% | 0.05% |

A significant better binding of CLRSGLGC phage to wells with immobilised FcFcR, compared to wells with no FcR activity, indicates that peptides selected from phage display libraries for binding INFγ stimulated U937 cells are recognised by FcγRI.

### Example 2

Identification of Poly IgR Binding Peptides

Library: The same libraries used for affinity selection for the FcγR in Example 1, were used, with SC (i.e. “secretory component”—the extracellular portion of the poly Ig receptor) as target.

Affinity selection: Free SC purified from human colostrum by Jackaline-Sepharose column (Pharmacia Biotech, Uppsala, Sweden) (Brandtzaeg P. Scand. J. Immunol. 1974, 3:579-88) was used. SC was immobilised in Nunc MaxiSorp (Costar) tubes at approximately 30 μg/ml in a volume of 500 μl in each tube at 4° C. ON. The tubes were blocked with 1% BSA or 1% milk powder in PBS for 1 h at RT. Approximately 10¹⁰ TU from each library were pre incubated with PBS and 1% BSA or 1% milk powder (1:1) for 1 h at 4° C. before they were added to the tubes and incubated at RT for 1.5 h. The tubes were washed 6 times with PBS/0.05% Tween 20. Bound phage were eluted by incubation with 500 μl 0.1M HCl-glycine pH 2.2 for 10 min and neutralised with 75 μl 0.1M Tris pH 9.1. Phage were amplified essentially as described by Smith and Scott (Methods Enzymol., 1993, 217:228-257) and two additional rounds of affinity selection were performed.

The output of phage increased from the first to the third round. The output from the C6-library increased from 3.0×10⁻⁴% to 5.0×10⁻¹%, while the output from the C9-library increased from 3.0×10⁻⁴% to 2.0×10⁻²%.

Single colonies of phage producing bacterial cells from the second and third round of selection were picked and expanded for further study.

ELISA assay. The purified colostrum SC was coated at 30 μg/ml in Nunc MaxiSorb wells in PBS at 4° C. ON. The wells were blocked with 200 μl PBS with 1% BSA or 1% milk powder for 1 h at RT. Phage supernatants from amplified eluates or single colonies were diluted 1:1 in blocking buffer, added to the wells and allowed to react with immobilsed SC for 1.5 h at RT. The wells were washed 6 times with PBS/0.05% Tween and bound phage were detected by adding a horse radish peroxidase (HRP)/Anti-M13 IgG conjugate (Pharmacia) 1:4000 in PBS with 1% BSA or 1% milk powder. After incubation for 1 h at RT, washing with PBS/0.605% Tween and addition of ABTS substrate solution (ABTS tablets from Boehringer Mannheim in citrate buffer, pH 4.0) the ABTS-HRP reactions were read in a microtiter plate reader set at 40⁵ nm (Dynatech MR 700).

The amplified eluates from three rounds of affinity selection showed an increase in amount of phage bound to immobilised SC (FIG. 3).

Single phage clones from the second and third round were also tested.

For the C9-library 50% of the clones were positive after the second round and 65% were positive after the third round. For the C6-library 30% of the clones were positive after the second round, and 95% were positive after the third round.

Sequencing of phage DNA demonstrated the presence of two dominating positive clones from each library. The sequences from the C6-library were cysteine rich and seemed to have double inserts of peptides: CQDPICFCGADGACYCTSRNC and CAWHYRFCGAAHSADGACREVFLVC (cysteines are underlined).

SC binding phage clones isolated from the C9-library displayed peptides with the sequence CVVWMGFQQVC or CWTSGARWRLC.

Transcytosis through human-pIgR-transfected MDCK cells: MDCK cells stably transfected with human pIgR were used to study transcytosis as described in Natvig et al., (J. Immunol., 1997, 159:4330-4340). Approximately 5.0×10⁵ cells were seeded on 3.0 μm collagen-coated PTFE filters (Transwell-COL 3494; Costar). The cell-layers were grown to confluence for 5-6 days at 37° C. with 5% CO₂ in DMEM (BioWhittaker; Walkersville, Md.) with 10% FCS (Life Technologies, Paisley, Scotland), 50 μg/ml gentamicin and 1 mM L-glutamine (Life Technologies). The filters were transferred to 200 μl medium including approximately 1.0×10⁸ TU of phage on the basolateral side. Then 200 μl medium were added apically and the cell layers incubated 16 h in 37° C. with 5% CO₂. The apical medium was then harvested, the filters washed in PBS and the membrane bound phage eluted in 200 μl 0.1M HCl, glycine pH2.2, 10 min at RT, before the cells were lysed in 200 μl lysis buffer (20 mM Tris pH 8.0 with 5 mM EDTA) for 10 min on ice. As a control for leakage through the cells 20 μg of IgG was added to the basolateral side and the amount on the apical side measured.

The amount of phage on the apical side was clearly higher for the positive phage clones compared to irrelevant clones, with an increase in transcytosis from about 1% for the negative phages to approximately 20% for the SC-binding phages.

Inhibition assay of SC binding phages: Nunc MaxiSorb 96 wells plates were coated with 30 μg/ml of purified colostrum SC at 4° C. ON. The plates were blocked in 1% milk powder for 1 h at RT before adding phage. Phage were pre incubated with 100 mM, 50 mM, 25 mM, 13 mM or 0 mM polymeric IgA (pIgA), pIgM or Streptococcus pneumoniae secretory IgA binding protein (SpsA) in PBS/1% milk powder. SpsA is an streptococcal produced SC binding protein (Hammerschmidt et al. Mol Microbiol 1997 sep 25 (6): 1113-24). After incubation for 1.5 h at RT the plates were washed 6 times before HRP/Anti-M13 conjugate (Pharmacia) 1:4000 in PBS with 1% milk powder was added. 6 times of washing with PBS/0.05% Tween were followed by the ABTS substrate solution. The ABTS-HRP reaction was read in a microtiter plate reader set at 405 nm. All samples were run in duplicates.

Positive phages from the C9-library (CWTSGARWRLC and CVVWMGFQQVC) were tested.

SpsA, but neither IgA nor IgM, blocked phage from binding to the receptor for both peptides. This suggests a common binding site for the phage clones and SpsA on SC (FIG. 4).

### Example 3

Generation and Characterisation of Pepbodies

Pepbodies are fusions between small antibody fragments that can be produced in E. coli (or by other means, known to those skilled in the art) and peptides with an ability to activate natural effector functions of the immune system. These peptides mimic the natural ligands of complement proteins and Fc receptors and are generally not parts of the natural ligand. Antibodies with affinity for the small hapten Nip have long been used as a models to study antibody effector functions (Sandlie and Michaelsen Mol Immunol. 1991 December; 28(12):1361-8. Review). Fusions of small antibody fragments with specificity for the hapten Nip and peptides that bind antibody effector ligands represent model systems to evaluate the effector activating potential of Pepbodies.

1. Generation of Antibody Fragments with Complement Activating Potential

Constructions of Single Chain Fv Antibody Fragments with Specificity for the Haptens NP and Nip.

The vector pLNOK (Norderhaug et al. J Immunol Methods. 1997 May 12;204(1):77-87.) contains the V_{H} NP/NIP fragment from the mammalian expression vector pSV2gptV_{NP} (Neuberger EMBO J. 1983;2(8):1373-8.) and PRO-145 (an expression vector of murine A1 light chain (Bebbington 1995 in Glover and Hames (Eds) IRL press Oxford 1995 page. 102), were used as sources for the V_{H} and V_{L} chains, respectively. Following amplification by PCR using the primers 5′ T TAC TCG CGG CCC AGC CGG CCA TGG CCC AGG TCC AAC TGC AGC AGC CTG G and 5′ TA GCG TAC CTC GAG-TGA GGA GAC TGT GAG AGT GGT GCC for the V_{H} fragment, and 5′ AT AGT CAA CTC GAG GGT GGT GGT GGT TCT GGG GGC GGA GGA TCC GGC GGG GGA GGG TCA GAG CTC CAG GCT GTT GTG ACT CAG GAA and 5′ TTT GTT CTG CGG CCG CAC CTA GGA CAG TCA GTT TGG T for the V_{L} fragment, the products were cut by restriction enzymes, ligated, PCR amplified and cloned into the bacterial expression vector pHOG21 (Kipriyanov et al J Immunol Methods. 1997 Jan. 15;200(1-2):69-77) as outlined in FIG. 5.

The anti Nip antibody fragments were expressed essentially as described (Kipriyanov et al J Immunol Methods. 1997 Jan. 15;200(1-2):69-77) and affinity purified with the hapten Nip coupled to sepharose 4B (Pharmacia, Sweden). Following concentration and desalting, the fragments were analysed by SDS Page and Western blotting (FIG. 6).

Creation of fUSE5 Compatible Cloning Sites.

The Myc/His6 tag of the pHOG vector (Kipriyanov et al 1997) (FIG. 5) was exchanged with an insert containing two Sfi1 sites creating the vector pSG1A (FIG. 7). The PSG1A vector is designed to have the same Sfi1 sites as found in the phage display vector fUSE5, this allows easy exchange of inserts between the two systems. A small double stranded DNA molecule (FIG. 7) was created to introduce the SfiI sites. The 70 bp long fragment was cut with NotI and XbaI. Biotinylated oligonucleotides were used in the PCR reaction allowing the subsequent removal of ends, uncut and partially cut inserts, by the addition of streptavidin followed by centrifugation trough a protein binding matrix (Centriflex™). The purified insert was ligated into NotI and XbaI cut vector.

Generation of Antibody Fragments with C-Terminal C1q Binding peptides.

Phage clones expressing C1q binding peptides (V. Lauvrak et al Biol. Chem. 1997 December; 378(12):1509-19) were used as the source of relevant DNA sequences. PCR products of ^{˜}350 bp were produced using the primers fUSE5-bio 5′TCGAAAGCAAGCTGATAAACCG and fUSE5-for bio 5′GTACAAACCACAACGCCTGTAG (FIG. 8).

The PCR products were cut with SfiI. Ends, uncut fragments and partially cut fragments were removed as described above. The products were then ligated into Sfi1 sites of pSG1A. These NP/Nip specific antibody fragments with C1q binding peptides (see table 5) as well as NP/Nip specific antibody fragments with the C-myc/Hi6 tag (M/H) were expressed in E. coli essentially as described in Kipriyanov et al. J Immunol Methods. 1997 Jan. 15;200(1-2):69-77.

TABLE 5
Sequences of the C-terminal part of anti-Nip
scFv fragments.

| | |
|---|---|
| M/H: | . . . . VLGAAAGSEQKLISEEDLNSHHHHHH-COOH |
| pSG1: | . . . VLGAAAADGAGSGAAGA-COOH |
| C10-1 | . . . . VLGAAAADGACRWDGSWGEVRCGAAGA-COOH |
| C10-2 | . . . . VLGAAAADGACYWVGTWGEAVCGAAGA-COOH |

M/H: c-myc/His₆-tag;
pSG1: vector with sfil sites with no insert;
C10-1: pSG1 with insert derived from C1q binding peptide C10-1,
C10-2; pSG1 with insert derived from C1q binding peptide C10-2.
(Inserts shown in bold).

SDS gels and Western blots revealed the antibody fragments to be present both in the periplasmic space and in the clarified growth medium (supernatant) (not shown.).

Antigen and C1q Binding

ELISA assays were used to evaluate the antigen (Nip), as well as the C1q binding capacity of the antibody fragments M/H, C10-1 and C10-2 (see Table 5). Nunc MaxiSorp wells were coated with 200 μl 1 μg/ml BSA/Nip as previously described (ref), and blocked for 1 h at RT with 1% BSA in PBS pH 7.4. Dilutions of supernatant in PBS were added to the wells and incubated for 1 h at RT. The wells were washed 10 times with PBS/0.05% Tween 20. To detect the binding of antibody fragments to Nip (FIG. 9A), a 1:500 dilution of biotinylated goat anti mouse λ-light chain (Gamλ-SOUTHERN BIOTECHNOLOGY ASSOCIATES, INC) was added. Incubation was continued for 1 hour at RT. The wells were washed as above and HRP conjugated streptavidin was added. The incubation was then continued for another hour at RT followed by washing as above. ABTS (Sigma) was added as a substrate for the HRP. To detect C1q binding to the immobilsed antibody fragments, 2 μg/ml C1q (CALBIOCHEM®) in PBS was added. After 1 h incubation at RT the wells were washed as above. Rabbit anti human C1q polyclonal serum (RAH-C1q-DAKO) diluted 1:6000 was added for 1 h RT. The wells were washed and 1:4000 diluted HRP conjugated sheep anti rabbitt (SAR) (Amersham) was added. After 1 h at RT the wells were washed again, and ABTS was added as described above (FIG. 9B).

Complement Fixation and Activation Potential.

The complement fixation-(C1q-binding) (FIG. 10) and activation-(FIG. 11) potential of the bacterial produced antibody fragments were analysed by ELISA assays with dilutions of Normal Human Serum (NHS) as the complement source. Nunc MaxiSorp wells were coated with BSA/Nip as described above. Dilutions of supernatants were added to the wells and allowed to bind Nip. Human IgG with Nip specificity was used as a positive control. The wells were washed once with veronal buffer (VB) followed by addition of NHS in VB. The complement fixation-(C1q binding) potential was analysed as described above using RAH anti-C1q. Deposition of C3b is an indication of complement activation and this was analysed by the use of rabbit anti C3b and HRP conjugated SAR as described for C1q binding.

Antibody fragments with C-terminal peptide fusions were expressed. The antibody fragments with C-terminal fusions retained their antigen binding capacity after bacterial expression. Peptides selected for binding C1q as phage protein III fusions retained their C1q binding activity also as fusions to small antibody fragments. In contrast to antibody fragments with a c-myc his-tag, antibody fragments with C1q binding peptides were able to activate complement and thus act as a Pepbody.

### Example 4

2. Generation of Fab Antibody Fragments with Complement Activating Potential

Construction of Fab Antibody Fragment with a Peptide Insertion in the f-g Loop (loop 6) in the Human IgG1 Constant Region, CH1.

A Plasmid pFab SfiI L6 was generated (FIG. 13). The way in which this plasmid was constructed is outlined in FIG. 15. Briefly, panel A shows the original Fab plasmid (derived from the vector used to express the single chain antibody fragments described earlier in these Examples) which encodes a Fab antibody fragment (in this case an anti phOx Fab). Panel B shows the step of PCR SOEing (Splicing by Overlap Extension) by the four oligonucleotides

| | | |
|---|---|---|
| 1: | 5′ Bio CATCCGCCCCAAAGCTTGCCTCCACC 3′, | +TL, 32 |
| 2: | 5′ GGCCCCAGCGGCCCCGGATCCGGCCCCGTCGGCCCCGGGCTTGT | |
| | GATTCACGTTGCAGATG 3′, | |
| 3: | 5′ GGGGCCGACGGGGCCGGATCCGGGGCCGCTGGGGCCAGCAACAC | |
| | CAAGGTGGACAAGAAAG 3′ and | |
| 4: | 5′ Bio TATAATAGGATCCCCCACAGTCTCCCCTGTTGAAGCT 3′ | |

Oligonucleotides 2 and 3 introduce a new SfiI site whereas the two biotinylated oligonucleotides 1 and 4 generate a larger biotinylated PCR fragment. The biotinylated PCR fragment was digested with HinDIII and NotI and ligated into an identical digested original Fab plasmid, thereby including the SfiI site into the region of the CH1 loop 6. Panel C Shows the final plasmid construct pFab SfiI L6 which encodes an anti phOx Fab. Into the SfiI sites DNA encoding different peptides can be inserted (see below). The DNA construct was verified by SfiI, BamHI digestion and confirmed by DNA sequencing.

It will be appreciated that any appropriate nucleic acid sequences which encode appropriate domains making up a Fab antibody fragment can be used in the vector shown in FIG. 13. In the specific example outlined below nucleic acid sequences encoding a phOx (2-phenyloxazol-5-one) Fab antibody were included in the vector.

Verification of Expression of Functional Fab Fragments from the pFab SfiX L6 Plasmid.

Anti phOx Fab SfiI L6 was expressed in E. Coli XL-1 blue essentially as described in Kiprianov et al. J. Immunol. Methods. 1997 Jan. 15; 200 (1-2):69-77. Microtiter plates (Maxisorp, NUNC) were coated with 200 μl 10 μg/ml BSA phOx. Supernatant from expressed anti phOx Fab SfiI L6 fragments was preincubated 1:1 in PBS with 1% BSA followed by incubation for 1 hour at RT in the plates. Bound Fab fragments were detected by rabbit anti Human kappa (Dako) and HRP conjugated donkey anti rabbit (Amersham). The signal was developed by ABTS and read at 405 nm (data not shown).

Insertion of C1q Binding Peptides in CH1 Loop 6 of Fab Fragments.

DNA encoding the C1q binding peptide CYWVGTWGEAVC was amplified by PCR, cut with Sfi1 and ligated into a Sfi1 digested anti-phOx Fab SfiI L6 plasmid. Single colonies were picked and inoculated into XL-1 blue for expression. The cultures were incubated at 35° C. overnight (ON) and expression was induced by removal of glucose and further incubation at 28° C. overnight.

Expression ELISA

Microtiter plates (Maxisorp, NUNC) were coated with 200 μl 10 μg/ml BSA pHOx. Supernatant from expressed Fab fragments was pre-incubated 1:1 in PBS with 1% BSA followed by incubation in the plates for 1 hour at RT. Bound Fab fragments were detected by rabbit anti Human kappa (Dako) and HRP conjugated donkey anti rabbit (Amersham). The signal was developed by ABTS and read at 405 nm (see FIG. 14).

C1q Binding ELISA.

Microtiter plates were coated and incubated as above. 5 μg/ml C1q was added. C1q bound to Fab fragments was detected by rabbit anti-Human C1q (Dako) (1:1000) and donkey anti-rabbit HRP (Amersham) (1:2000). The signal was developed by ABTS and read at 405 nm (see FIG. 14).

1. A binding molecule comprising (i) one or more polypeptides which form a binding site capable of binding a target molecule and (ii) an Fc effector peptide displaying one or more effector functions associated with the constant region (Fc) of an immunoglobulin heavy chain.

2. The binding molecule of claim 1 wherein the polypeptides forming the binding site are derived from an antibody molecule or a derivative thereof.

3. The binding molecule of claim 1 or claim 2 wherein the binding site comprises an antibody fragment.

4. The binding molecule of claim 3 wherein the antibody fragment is a ScFv, Fv or Fab fragment.

5. The binding molecule of any one of claims 1 to 4 wherein the Fc effector peptide has the ability to bind Fc-receptors and/or the ability to activate complement.

6. The binding molecule of claim 5 wherein the Fc effector peptide has the ability to activate complement and binds to the C1q protein.

7. The binding molecule of claim 6 wherein the Fc effector peptide comprises one or more of the amino acid sequences CRWDGSWGEVRC or CYWVGTWGEAVC, or functional fragments thereof, or a sequence which is substantially homologous to these sequences or fragments.

8. The binding molecule of claim 6 wherein the Fc effector peptide comprises one or more of the amino acid sequences h/RWXXXWG or R/KP/DCPS/TCPXXP (where h is a large hydrophobic amino acid, X is a less conserved amino acid and underlined residues are invariant amino acids), or functional fragments thereof, or a sequence which is substantially homologous to these sequences or fragments.

9. The binding molecule of claim 5 wherein the Fc effector peptide has the ability to bind Fc-receptors and comprises the amino acid sequence CLRSGXXC (where X is a variable amino acid).

10. The binding molecule of claim 9 wherein the Fc effector peptide comprises one or more of the amino acid sequences CLRSGRGC, CLRSGLGC, CLRSGAGC, CLRSGSGC, CLRSGRAC, CLRSGANC, or CLRSGLHC, or functional fragments thereof, or a sequence which is substantially homologous to these sequences or fragments.

11. The binding molecule of claim 5 wherein the Fc effector peptide has the ability to bind Fc-receptors and comprises one or more of the amino acid sequences CRRSGQGC, CLYGDELC, CFPVGRATC, CSWIPGVGLVC, CRRATAGCAGC, CRSMVMLRVRC, CGRVNTWLPQC or CSAGRACCRYC, or functional fragments thereof, or a sequence which is substantially homologous to these sequences or fragments.

12. The binding molecule of claim 5 wherein the Fc effector peptide has the ability to bind Fc-receptors and comprises one or more of the amino acid sequences CQDPICFCGADGACYCTSRNC, CAWHYRFCGAAHSADGACREVFLVC, CVVWMGFQQVC or CWTSGARWRLC, or functional fragments thereof, or a sequence which is substantially homologous to these sequences or fragments.

13. The binding molecule of any one of claims 1 to 12 wherein said molecule comprises two or more different Fc effector peptides which exhibit the same or differing effector functions.

14. An Fc effector peptide which has the ability to bind one or more Fc-receptors.

15. The Fc effector peptide of claim 14, wherein said Fc effector peptides are as defined in any one of claims 9 to 12.

16. A nucleic acid molecule comprising nucleic acid sequences which encode one or more Fc effector peptides displaying one or more effector functions associated with the constant region (Fc) of an immunoglobulin heavy chain, or a nucleic acid molecule comprising nucleic acid sequences which are degenerate to, substantially homologous with or which hybridise with such nucleic acid sequences, or which hybridise with the sequence complementary to such an encoding sequence, or fragments thereof.

17. A nucleic acid molecule comprising nucleic acid sequences which encode one or more polypeptides which form all or part of a binding site capable of binding a target molecule, together with nucleic acid sequences which encode one or more Fc effector peptides displaying one or more effector functions associated with the constant region (Fc) of an immunoglobulin heavy chain, or a nucleic acid molecule comprising nucleic acid sequences which are degenerate to, substantially homologous with or which hybridise with such nucleic acid sequences, or which hybridise with the sequence complementary to such an encoding sequence, or fragments thereof.

18. The nucleic acid molecule of claim 16 or claim 17 wherein said Fc effector peptides are as defined in any one of the preceding claims.

19. An expression vector comprising the nucleic acid molecules as defined in any one of claims 16 to 18.

20. Host cells expressing the nucleic acid molecules as defined in any one of claims 16 to 18 or containing an expression vector as defined in claim 19.

21. A method of producing the binding molecules as defined in any one of claims 1 to 13, comprising the steps of (i) the expression in a host cell of a nucleic acid molecule encoding one or more polypeptides which form all or pert of a binding site capable of binding a target molecule and one or more Fc effector peptides displaying one or more effector functions associated with the constant region (Fc) of an immunoglobulin heavy chain and (ii) the isolation of the expressed binding molecules from the host cells or from the supernatant.

22. A method of producing an Fc effector peptide as defined in any one of claims 14 to 15 comprising the steps of (i) growing a host cell containing a nucleic acid molecule encoding an Fc effector peptide displaying one or more effector functions associated with the constant region (Fc) of an immunoglobulin heavy chain under conditions suitable for the expression of the Fc effector peptide; and (ii) isolating the Fc effector peptide from the host cell or from the supernatent.

23. The binding molecules or the Fc effector peptides as defined in any one of the preceding claims for use in therapy, diagnosis or imaging.

24. Use of the binding molecules or the Fc effector peptides as defined in any one of the preceding claims in the manufacture of a composition for use in therapy, imaging or diagnosis.

25. A method of treatment of a subject comprising the administration of an appropriate amount of a binding molecule or an Fc effector peptide as defined in any one of the preceding claims to a subject, or to a sample removed from a subject and which is subsequently returned to the subject.

26. A method of diagnosis or imaging of a subject comprising the administration of an appropriate amount of a binding molecule as defined in any one of claims 1 to 13 to the subject and detecting the presence and/or amount of the binding molecule in the subject.

27. Pharmaceutical compositions comprising the binding molecules or the Fc effector peptides as defined in any one of the preceding claims, together with one or more pharmaceutically acceptable carriers or excipients

28. A reagent which comprises a binding molecule or an Fc effector peptide as defined in any one of the preceding claims.

29. Use of a binding molecule or an Fc effector peptide as defined in any one of the preceding claims to induce Fc receptor functions.

30. A kit comprising a binding molecule or an Fc effector peptide as defined in any one of the preceding claims.
